SEQUENCE LISTING (187 sequences)

SEQ ID NO: 1 - protein, 119 aa, Rabbit
QSVEESGGRLVTPGTPLTLTCTVSGIDLSSYAMSWVRQAPGKGLEWVGIIRGSGSIYYANWAKGRFTISKTSTTVDLRFASPTTEDTATYFCARVLWPGSVAYAYHNIWGPGTLVTVSS

SEQ ID NO: 2 - protein, 119 aa, Rabbit
QSVEESGGRLVTPGTPLTLTCTVSGIDLNSYAMSWFRQAPGKGLEWIGLIRRSGATYYASWAKGRFTISKASTTVDLKFTSPTTEDTATYFCARVLWPGDIAYAYHNIWGPGTLVTVSS

SEQ ID NO: 3 - protein, 119 aa, Rabbit
QSVEESGGRLVTPGTPLTLTCTVSGIDLNNYAMSWFRQAPGKGLEWIGLIRSSGATYYASWAKGRFTISKASTTVDLKFTSPTTEDTATYFCARVLWPGDIAYAYHNIWGPGTLVTVSA

SEQ ID NO: 4 - protein, 119 aa, Rabbit
QSVEESGGRLVTPGTPLTLTCTVSGFSLSSYAMSWFRQAPGKGLEWIGLIRSSGATYYASWAKGRFTISKASTTVDLKFTSPTTEDTATYFCARVLWPGDIAYAYHNIWGPGTLVTVSS

SEQ ID NO: 5 - protein, 119 aa, Rabbit
QSVEESGGRLVTPGTPLTLTCTVSGFSLSTYAMSWFRQAPGKGLEWIGLIRSSGATYYASWAKGRFTISKASTTVDLKFTSPTTEDTATYFCARVLWPGDIAYAYHNIWGPGTLVTVSS

SEQ ID NO: 6 - protein, 119 aa, Rabbit
QSVEESGGRLVTPGTPLTLTCTVSGFSLSNYAMSWFRQAPGKGLEWIGLIRSSGATYYASWAKGRFTISKASTTVDLKFTSPTTEDTATYFCARVLWPGDIAYAYHNIWGPGSLVTVSS

SEQ ID NO: 7 - protein, 119 aa, Rabbit
QSVEESGGRLVTPGTPLTLTCTVSGFSLNNYAMSWFRQAPGKGLEWIGLIRSSGATYYASWAKGRFTISKASTTVDLKFTSPTTEDTATYFCARVLWPGDIAYAYHNIWGPGTLVTVSS

SEQ ID NO: 8 - protein, 119 aa, Rabbit; identical to SEQ ID NO: 7

SEQ ID NO: 9 - protein, 119 aa, Rabbit; identical to SEQ ID NO: 7

SEQ ID NO: 10 - protein, 119 aa, Rabbit
QSVEESGGRLVTPGTPLTLTCTVSGFSLNNYAMSWFRQAPGKGLEWIGLIRSSGAAYDAPWAKGRFTISKASTTVDLKFTSPTTEDTATYFCARVLWPGDIAYAYHNIWGPGTLVTVSS

SEQ ID NO: 11 - protein, 119 aa, Rabbit
QSLEESGGRLVTPGTPLTLTCTVSGFSLNNYAMSWFRQAPGKGLEWIGLIRSGGATYYASWAKGRFTISKVSTTVDLKFTSPTTEDTATYFCARVLWPGDIAYAYHNIWGPGTLVTVSS

SEQ ID NO: 12 - protein, 119 aa, Rabbit
QSLEESGGRLVTPGTPLTLTCAVSGIDLSSYAMSWFRQAPGKGLEWIGLIRSSGATYYASWAKGRFTISKASTTVDLKMTSLTTEDTATYFCARVLWPGDIAYAYHNIWGPGTLVTVSS

SEQ ID NO: 13 - protein, 119 aa, Rabbit
QSLEESGGRLVTPGTPLTLTCTVSGIDLSAYAMSWVRQAPGKGLEWIGLIRSSGATYYASWAKGRFTISKTSTTVDLKMTSPTTEDTATYFCARVLWPGEIAYAYHNIWGPGTLVTVSS

SEQ ID NO: 14 - protein, 119 aa, Rabbit
QSVEESGGRLVTPGTPLTLTCTVSGIDLNNYAMSWVRQAPGKGLEWIGIIGSSGSIFYASWAKGRITISKTSTTVDLKFTSLTTEDTATYFCARVLWAGSVAYAYHNIWGPGTLVTVSS

SEQ ID NO: 15 - protein, 119 aa, Rabbit
QSVEESGGRLVTPGTPLTLTCTVSGFSLSSYAMSWVRQAPGKGLEWIGIISSSGNTYFASWAKGRFTISKTSTTVDLKFTSPTTEDTATYFCARVLWAGSVAYAYHNIWGPGTLVTVSS

SEQ ID NO: 16 - protein, 119 aa, Rabbit
QSVEESGGRLVTPGTPLTLTCTVSGFSLSSYAMSWVRQAPGKGLEWIGIIRPGGNTYSASWAKGRFTISKTSTTVDLKFTSPTPEDTATYFCARVLWAGDVAYAYHNIWGPGTLVTVSS

SEQ ID NO: 17 - protein, 119 aa, Rabbit
QSVEESGGRLVTPGTPLTLTCTVSGFSLSDYAMSWVRQAPGKGLEWIGMVRDTGVTFYAGWAKGRFTISKTSTTVDLKFTSPTTEDTATYFCARVLWAGYVAYAYHNIWGPGTLVTVSS

SEQ ID NO: 18 - protein, 119 aa, Rabbit
QSVEESGGRLVTPGTPLTLTCTASGFSLSDYAMSWVRQAPGKGLEWIGIISNSGITFYAGWAKGRFTISKTSTTVDLKFTSPTTEDTATYFCARALWAGYIAYVYHNIWGPGTLVTVSS

SEQ ID NO: 19 - protein, 119 aa, Rabbit
QSVEESGGRLVTPGTPLTLTCTASGFALNDFAMSWVRQAPGKGLEWIGMIASSGNTFYASWAKGRFTISKTSTTVDLKFTSPTTEDTATYFCARVLWPGYIAYAYHNIWGPGTLVTVSS

SEQ ID NO: 20 - protein, 116 aa, Rabbit
QSLEESGGRLVTPGTPLTLTCTVSGIDLSSYAMGWVRQAPGKGLEHIGIITASGGIYYASWAKGRFTISKTSTTVDLRIPSPTTEDTGTYFCARTENSYFLYFTIWGPGTLVTVSS

SEQ ID NO: 21 - protein, 119 aa, Rabbit
QSLEESGGRLVTPGTPLTLTCTVSGIDLSSYAMGWVRQAPGKGLEWIGFISSSGSTYYASWAKGRFTISKTSTTVDLKITSPTTEDTATYFCANNYDDYGDFLHYFNIWGPGTLVTVSS

SEQ ID NO: 22 - protein, 110 aa, Rabbit
QSVEESGGRLVTPGTPLTLTCTVSGIDLSSNAMAWVRQAPGKGLEWIGIIDGDVSPYYASWAKGRFTISKTSSTTVDLKMTSLTTEDTATYFCARPFNIWGPGTLVTVSS

SEQ ID NO: 23 - protein, 116 aa, Rabbit
QSVEESGGRLVTPGTPLTLTCTVSGIDLSKNAISWVRQVPGEGLEWIGIIYGDGNRDYANWAKGRFTISKTSTTVDLKVTSPTTEDTATYFCARGTTIWSDYLDIWGPGTLVTISS

SEQ ID NO: 24 - protein, 119 aa, Rabbit
QSVEESGGRLVTPGTPLTLTCTVSGFSLSSNAMSWVRQAPGEGLEWIGTISSSGSTYYATWVKGRFTISKTSTTVDLKMTSLTAADTATYFCARGDDDVSDYFYYFPIWGPGTLVTVSS

SEQ ID NO: 25 - protein, 119 aa, Rabbit; identical to SEQ ID NO: 24

SEQ ID NO: 26 - protein, 119 aa, Rabbit; identical to SEQ ID NO: 24

SEQ ID NO: 27 - protein, 119 aa, Rabbit; identical to SEQ ID NO: 24

SEQ ID NO: 28 - protein, 119 aa, Rabbit; identical to SEQ ID NO: 24

SEQ ID NO: 29 - protein, 117 aa, Rabbit
QSLEESGGRLVTPGGSLTLTCTASGFSLTSYWMSWVRQAPGKGLEWIGAVSNSGTTYYASWAKGRFTISRTSTTVDLRMTRLTTEDTATYFCARDAGDNYFTWLDLWGQGTLVTVSS

SEQ ID NO: 30 - protein, 121 aa, Rabbit
EEQLKESGGDLVKPEGSLTLTCKASGFDLSSEFYICWVRQAPGKGLEWIGCIATVSSRRLYASWVNGRFTISKTSSTTVTLQMPSLTAADTATYFCARDDSARNWFYFYLWGPGTLVTVSS

SEQ ID NO: 31 - protein, 118 aa, Rabbit
QSLEESGGDLVKPGTSLTLTCTASGFSFSARHFMYWVRQAPGKGLEWIGCIDIGSGSTYYTSWAKDRFTISKPSSTTVTLQMTSLTAADTATYFCARSSGYPYYFTLWGPGTLVTVSS

SEQ ID NO: 32 - protein, 122 aa, Rabbit
QQQLVESGGGLVKPGASLTLTCKASGFPVTHWWMCWVRQAPRKGLELIACAYTGDLTTYHASWAIGRFTISTSSSTMVTLQMTSLTVADTATYFCATWGAHNQGYWDGFDPWGPGTLVTVSS

SEQ ID NO: 33 - protein, 122 aa, Rabbit
QEQLVESGGDLVQPEGSLTLTCKASGLDFSSSYWICWVRQAPGKGLELIACIYTDSGGIWYTSWAKGRFTISRSTSLNTVDLKVSSLTAADTATYFCARNYAGYSSGIFNLWGPGTLVTVSS

SEQ ID NO: 34 - protein, 125 aa, Rabbit
QEQLEESGGGLVKPEGSLTLTCTASGFSFISNYWMCWVRQAPGKGLEWIACIYAGGGISTYYASWAKGRFTISKTSSTTVTLQMTSLTAANTATYFCARAYVYSGAYLYYGMDLWGPGTLVTVSS

SEQ ID NO: 35 - protein, 122 aa, Rabbit
QSLEESGGDLVKPGASLTLTCTASGFSASSSYWMCWVRQAPGKGLELIACTYSSSGNTNYASWAKGRFTSSITSSTTVTLQMASLTAADTATYFCARDNYDDHGAWLYFNLWGPGTLVTVSS

SEQ ID NO: 36 - protein, 123 aa, Rabbit
QSLEESGGDLVKPGASLTLTCTASGFSFSSNYYMCWVRQAPGKGPEWIACIYGGSIGDPSYASWAKGRFTISKASSTTVTLQMTSLTAADTATYFCAREEVGVSAPSRGWGLWGPGTLVTVSS

SEQ ID NO: 37 - protein, 121 aa, Rabbit
QSLEESGGDLVKPGASLTLTCTASGFSFSSGYYMCWVRQAPGKGLEWIACIGVSTQGAYYASWTEGRFTISKTSSTTVTLQMTSLTAADTATYFCARTAGAPADSLYFTLWGPGTLLTVSS

SEQ ID NO: 38 - protein, 119 aa, Rabbit
QSSEESGGDLVKPGASLTLTCTASGFSLSSGYDMCWVRQAPGKGLEWIGCIKTGATNEYYASWAKGRFTISRTSSTTVTLRMTSLTAADTATYFCAREDTNNWGSLNLWGPGTLVTVSS

SEQ ID NO: 39 - protein, 116 aa, Rabbit
QSVEESGGRLVTPGTPLTLTCTVSGFSLGSFAVGWVRQPPGEGLEWIGLINADEARYYASWAKGRFTISRTSTTVDLRITSPTIEDTATYFCARAPDNFFYYFSMWGPGTLVTVSS

SEQ ID NO: 40 - protein, 116 aa, Rabbit; identical to SEQ ID NO: 39

SEQ ID NO: 41 - protein, 119 aa, Rabbit
QSVEESGGRLVTPGTPLTLTCSVSGFSLNTYAVIWVRQAPGKGLEYIGVVAGGGYIYFTTWAKGRFTISKTSTTVDLKMTSPTTEDTATYFCARGDVDDNADYSRLDIWGPGTLVTVSL

SEQ ID NO: 42 - protein, 116 aa, Rabbit
QSVEESGGRLVTPGTPLTLTCTVSGFSLNNYAMGWFRQAPGKGLEWIGLIYVSGITSYASWVNGRFTISKTSTTVDLKMTSLTVSDTATYFCARDLYGGDHYYIIWGPGTLVTVSS

SEQ ID NO: 43 - protein, 116 aa, Rabbit
QSMEESGGRLVTPGTPLTLTCTVSGVDLSIYAMSWVRLAPGKGLEWIGVILSSGRSVYTSWAKGRFTISKTSTTVDLKMTSPTIEDTATYFCARGYDDYVALFNMWGPGTLVTVSS

SEQ ID NO: 44 - protein, 116 aa, Rabbit
QSMEESGGRLVTPGTPLTLTCTVSGFDLSIYAMSWVRQAPGKGLEWIGVILSSGRSVYAGWAKGRFTISKTSTTVDLKITSPTIEDTAAYFCARGYDDYVALFNMWGPGTLVTVSS

SEQ ID NO: 45 - protein, 116 aa, Rabbit
QSVEESGGRLVTPGTPLTLTCTVSGIDLSSYAMSWVRQAPGKGLEWIGIISSTGNTCYANWAKGRFTISKTSTTVDLKITSPTTEDTATYFCARGYDDYVALFNMWGPGTLVTVSS

SEQ ID NO: 46 - protein, 116 aa, Rabbit
QSVEESGGRLVTPGTPLTLTCTVSGIDLSSYAMSWVRQAPGKGLEWIGIIFSSGNIVYARRAKGRFTISKTSTTVDLQITSPTIEDTATYFCARGYDDYVALFNMWGPGTLVTVSS

SEQ ID NO: 47 - protein, 116 aa, Rabbit
QSVEESGGRLVTPGGSLTLTCTVSGIDLSSYAMSWVRQAPGKGLEWIGIITSRAITYYATWAKGRFTISKTSTTVELKITSPTTEDTATYFCARGYDDYVALFNMWGPGTLVTVSS

SEQ ID NO: 48 - protein, 110 aa, Rabbit
DPVLTQTPASVSEPVGGTVTIKCQASEDLGSNLAWYQQKPGQPPKLLIYSASTLTSGVPSRFKGSGSGTEYTLTISGVQCDDAATYYCQDTYYGNTYLGAFGGGTEVVVR

SEQ ID NO: 49 - protein, 110 aa, Rabbit
DLVMTQTPSPVSAAVGGTVTINCQASEEIGGNLAWYQQKPGQPPKLLIYSASTLTSGVPSRFKGSGFGTEYTLTISDLECDDAATYYCQYTYYGFTYVGPFGGGTEVVVK

SEQ ID NO: 50 - protein, 110 aa, Rabbit
DLVMTQTPSPVSAAVGGTVTINCQASEEIGGNLAWYQQKPGQPPQLLIYSASTLTSGVPSRFKGSGFGTEYTLTISDLECADAATYYCQYTYYGFTYVGPFGGGTEVVVK

SEQ ID NO: 51 - protein, 110 aa, Rabbit
DLVMTQTPSPVSAAVGGTVTINCQASEEIGGNLAWYLQKPGQPPQLLIYSASTLTSGVPSRFKGSGFGTEYTLTISDLECADAATYYCQYTYYGFSYVGPFGGGTEVVVK

SEQ ID NO: 52 - protein, 110 aa, Rabbit
DLVMTQTPSPVSAAVGGTVTINCQASEEIGGNLAWYQQKPGQPPQLLIYSASTLASGVPSRFKGSGFGTEYTLTISDLECADAATYYCQYTYYGFSYVGPFGGGTEVVVK

SEQ ID NO: 53 - protein, 110 aa, Rabbit
DLVMTQTPSPVSAAVGGTVTINCQASEEIGGNLAWYQQKPGQPPKLLIYSASTLTSGVPSRFKGSGFGTEYTLTISDLECADAATYYCQYTYYGFSYVGPFGGGTEVVVK

SEQ ID NO: 54 - protein, 110 aa, Rabbit; identical to SEQ ID NO: 53

SEQ ID NO: 55 - protein, 110 aa, Rabbit
DLVMTQTPSPVSAAVGGTVTINCQASEEIGDNLAWYQQKPGQPPKLLIYSASTLTSGVPSRFKGSGFGTEYTLTISDLECADAATYYCQYTYYGFSYVGPFGGGTEVVVK

SEQ ID NO: 56 - protein, 110 aa, Rabbit
DLVMTQTPSPVSAPVGGTVTINCQASEEIGGNLAWYQQKPGQPPKLLIYSASTLTSGVPSRFKGSGFGTEYTLTISDLECADAATYYCQYTYYGFSYVGPFGGGTEVVVK

SEQ ID NO: 57 - protein, 110 aa, Rabbit
DLVMTQTPSPVSAAVGGTVTINCQASEEIGGNLAWYQQKPGQPPKLLIYSASTLTSGVPSRFKGSGSGTEYTLTISDLECADAATYYCQYTYYGFSYVGPFGGGTEVVVK

SEQ ID NO: 58 - protein, 110 aa, Rabbit
DLVMTQTPSPVSAAVGGTVTINCQASEEIGGNLAWYQQKPGQPPKLLIYSASTLTSGVPSRFKGSGSGTEYTLTINDLECADAATYYCQYPYYGFSYVGPFGGGTEVVVK

SEQ ID NO: 59 - protein, 110 aa, Rabbit
DLVMTQTPSPVSAAVGGTVTINCQASEEIGGNLAWYQQKPGQPPKLLIYSASTLASGVPSRFKGSGSGTEYSLSISDLECADAATYYCQYTYYGFSYVGPFGGGTEVVVK

SEQ ID NO: 60 - protein, 110 aa, Rabbit
DVVMTQTPASVSEPVGGTVTINCQASEEIGSNLAWYQQKPGQPPKLLIYSASTLTSGVPSRFKGSGSGTEYTLTISDLECADAATYYCQYTYYGFNYVGPFGGGTEVVVK

SEQ ID NO: 61 - protein, 110 aa, Rabbit
DVVMTQTPASVSEPVGGTVTINCQASEEIASNLAWYQQKPGQPPKLLIYSASTLTSGVPSRFKGSGSGTEYTLTISDLECADAATYYCQYTYYGFNYVGPFGGGTEVVVK

SEQ ID NO: 62 - protein, 110 aa, Rabbit
DVVMTQTPASVSEPVGGTVTINCQASEEIGGNLAWYQQKPGQPPKLLIYSASSLTSGVPSRFKGSESGTEYTLTISDLECADAATYYCQYSYYGFNYVGPFGGGTEVVVK

SEQ ID NO: 63 - protein, 110 aa, Rabbit
GPVMTQTPASVSEPVGGTVTIKCQASEDIGSNLAWYQQKPGQPPNLLVYSASTLTSGVPSRFSGSGSGTEYTLTISDLECADAATYYCQDTYYGNTYLGAFGGGTEVVVK

SEQ ID NO: 64 - protein, 110 aa, Rabbit
GPVMTQTPASVSEPVGGTVTMKCQASEDIGSNLAWYQQKPGQPPNLLVFSASTLASGVPSRFAGSGSGTEYTLTISDLECADAATYYCQDSYYGNSYLGAFGGGTEVVVK

SEQ ID NO: 65 - protein, 110 aa, Rabbit
DPVMTQTPASVSEPVGGTVTINCQASEELGSNLAWYQQKPGQPPKLLIYSASTLASGVPSRFKGSGSGTEYTLTISDLECADAATYYCQYTYFGSSYLGAFGGGTEVVVK

SEQ ID NO: 66 - protein, 110 aa, Rabbit
DPVLTQTPASVSAVVGGTVTINCQTSEDIASNLAWYQQKSGQRPKLLIYEASKLASGVPSRFKGSGSGTEYTLTISDLECADAATYYCQYTYYGSSYLGAFGGGTEVVVK

SEQ ID NO: 67 - protein, 111 aa, Rabbit
AVVLTQTASPVSGAVGGTVTIKCQASQNIYNNLAWYRQKPGQPPKLLIYAASKLASGVPSRFSGSRSGTQFTLSISDLECADAATYYCQSYYFDSSSTDAAFGGGTEVVVK

SEQ ID NO: 68 - protein, 111 aa, Rabbit; identical to SEQ ID NO: 67

SEQ ID NO: 69 - protein, 111 aa, Rabbit; identical to SEQ ID NO: 67

SEQ ID NO: 70 - protein, 111 aa, Rabbit; identical to SEQ ID NO: 67

SEQ ID NO: 71 - protein, 111 aa, Rabbit; identical to SEQ ID NO: 67

SEQ ID NO: 72 - protein, 111 aa, Rabbit
AVVLTQTASPVSGAVGGTVTIKCQASQHIYSNLAWYQQKPGQRPKLLIYAASKLASGVPSRFSGSRSGTEFTLTISDLECADAATYYCQSYYYSSDSTDNTFGGGTEVVVK

SEQ ID NO: 73 - protein, 111 aa, Rabbit
AVVLTQTPSSVSAAVGGTVTINCQASENIYSNLAWYQQKPGQPPKLLIYGASALPSGVPSRFSGSRSGTQFTLTISDLECADAATYYCQSYFYSSSNDDNPFGGGTEVAVK

SEQ ID NO: 74 - protein, 112 aa, Rabbit
AVVLTQTTSSVSADVGGTVTIKCQASENIYSLLAWYQQKPGQPPKLLIYSASDLASGVPSRFSGSGFGTEFTLTISDLECADAATYHCQSYYYSGSSADTGAFGGGTEVVVK

SEQ ID NO: 75 - protein, 111 aa, Rabbit
AVVLTQTASPVSGAVGGTVTINCQASQNIYSNLAWYQHKPGQPPKLLIYKASTLASGVPSRFKGSGSGTEYILTISDLECADAATYYCQTNYYSINGGEVTFGGGTEVVVK

SEQ ID NO: 76 - protein, 110 aa, Rabbit
DVVMTQTPASVSEPVGGTVTINCQASQSIRLSDLAWYQQKPGHPPKLLIYKASKLASGVPSRFKGSGSGTEYTLTISGVQCDDAATYYCQSIDYDNYVFFFGGGTEVVVK

SEQ ID NO: 77 - protein, 110 aa, Rabbit
ALVMTQTPASVSAAVGGTVTISCQASQSIHSWVSWYQQKPGQPPKLLIYYAATLASGVPSRFKGSGSGKQFTLTISDLECDDAATYYCQQDFGGSDVDNTFGGGTEVVVA

SEQ ID NO: 78 - protein, 112 aa, Rabbit
QVLTQSPSPVSAAVGGTVTINCQASQSVYNGDWLGWYQQKPGQPPKLLIYDASTLASGVSSLINCNGSGTQWTLTISGVQCDDAATYYCQGEFSCSSADCVAFGGGTEVVVK

SEQ ID NO: 79 - protein, 113 aa, Rabbit
ADIVLTQTPASVSATVGGTVTIKCQASQNIYSNLAWYQQRPGQPPKLLIYAASNLASGVSSRFSGSRSGTEFTLTISDLECADAATYYCQSYYYSSDDNYLNTFGGGTEVVVK

SEQ ID NO: 80 - protein, 113 aa, Rabbit; identical to SEQ ID NO: 79

SEQ ID NO: 81 - protein, 107 aa, Rabbit
QVLTQTPASVSATVGGTVTIKCQASQNIYSNLAWYQQTPGQPPKLLIYGASNLASGVPSRFKGSGSGTQFTLTISDLECADAGTYYCQDYYGTSRYIFGGGTEVVVG

SEQ ID NO: 82 - protein, 111 aa, Rabbit
ADIVMTQTPASVSEPVGGTVTIKCQASQSIYNYLSWYQQKPGQPPKLLIYAASNLASGVPSRFKGSGSGTQFTLTISDLECADAATYYCQSNYGTSSASYAFGGGTEVVVK

SEQ ID NO: 83 - protein, 107 aa, Rabbit
DVVMTQTPFSVSAAVGGTVTINCQASESIYINLAWYQQKPGQPPKLLIYDASNLASGVPSRFKGSGSGTEYTLTISDLECADATTYYCQNYYGSSYDFGGGTEVVVK

SEQ ID NO: 84 - protein, 111 aa, Rabbit
DVVMTQTPASVEAAVGGTVTINCQASENIYSGLAWYQQKPGQPPKLLIYDASNLETGVPSRFKGSGSGAQFTLTISGVQCDDAATYYCQCSEYDSSYVPNVFGGGTEVVVK

SEQ ID NO: 85 - protein, 107 aa, Rabbit
QVLTQTPASVSAAVGGTVTIKCQASESVSSRLAWYQQKAGQPPKLLIYSASTLASGVSSRFKGSGSGTEFSLTISDLECADAATYYCQSYYRIDRNLFGEGTEVVVK

SEQ ID NO: 86 - protein, 110 aa, Rabbit
AVVLTQTPASVSAAVGGTVTINCQASQSINSGLAWYQQKPGQPPKLLIYQASTLASGVPSRFKGSGSGTEYTLTISDLECADAATYYCQQTYDTSDLDNAFGGGTEVVVK

SEQ ID NO: 87 - protein, 110 aa, Rabbit
AYDMTQTPASVEVAVGGTVTIKCQASEDIESYLAWYQQKPGQRPLLLIYAASTLASGASSRFKGSGSGTEFTLTISGVECADAATYYCHQAYTTTGLDAGFGGGTEVVVK

SEQ ID NO: 88 - protein, 111 aa, Rabbit
IVMTQTPSSKSVPVGDTVTINCQASESVYNGDRLAWFQQKPGQPPKRLIYLASTLASGVPSRFKGSGSGTQFTLTISDVVCADAATYYCAGYRSASTDGNAFGGGTEVVVK

SEQ ID NO: 89 - protein, 110 aa, Rabbit
AVLTQTPSPVSAAVGGTVTISCQSSKSVYADNCLAWYQQKPGQRPRLLIYEASKLPSGVPSRFKGSGSGTQFTLTISDVQCDDAATYYCAGDYSPTSDNSFSGGTEVVVK

SEQ ID NO: 90 - protein, 110 aa, Rabbit
AVLTQTPSPVSAAVGGTVTIKCQSSQSVYNHNLLSWYQHKPGQPPKLLIYDASTLASGVPSRFKGNGSGTQFTLTISGVQCADAATYYCLGGYDDDADTAFGGGTEVVVR

SEQ ID NO: 91 - protein, 110 aa, Rabbit
AVLTQTPSPVSAAVGGTVTIKCQSSQSVYNNNLLSWYQHKPGQPPKLLIYDASTLASGVPSRFKGSGSGTQFTLTISGVQCADAATYYCLGGYDDDADTAFGGGTEVVVR

SEQ ID NO: 92 - protein, 110 aa, Rabbit
AVLTQTPSPVSAAVGGTVTIKCQSSQSVYNNALLSWYQQKSGQPPKLLIYDASKLASGVPDRFSGSGSGTQFTLTISGVQCDDAATYYCLGGYDDDADTTFGGGTEVVVE

SEQ ID NO: 93 - protein, 110 aa, Rabbit
AVLTQTPSPVSAAVGGTVTIKCQSSQSVYNNNALSWYQQKPGQPPKLLIYDASTLASGVPDRFSGSGSGTQFTLTISGVQCDDAATYYCLGGYDDDADTAFGGGTEVVVK

SEQ ID NO: 94 - protein, 110 aa, Rabbit
AVVTQTPSPVSATVGGTVTISCQSSESVYNDVCLSWYQQKPGQRPKLLIYDAFTLASGVSSRFKGSGSGTQFTLTISGVQCDDAATYYCLGGYDDDADTAFGGGTEVVVK

SEQ ID NO: 95 - protein, 17 aa, Rabbit
CARDINSYGYAYATDIW

SEQ ID NO: 96 - protein, 14 aa, Rabbit
CARGYAGSSYYNLW

SEQ ID NO: 97 - protein, 15 aa, Rabbit
CARSDYSYGGAYDIW

SEQ ID NO: 98 - protein, 13 aa, Rabbit
CARRVDSTGTDIW

SEQ ID NO: 99 - protein, 10 aa, Rabbit
CGSGYYINIW

SEQ ID NO: 100 - protein, 17 aa, Rabbit
CARGGAGISGYTYFNIW

SEQ ID NO: 101 - protein, 14 aa, Rabbit
CARGCPGYGDNDIW

SEQ ID NO: 102 - protein, 11 aa, Rabbit
CARGYWSLDIW

SEQ ID NO: 103 - protein, 15 aa, Rabbit
CVRDSTGISALFNVW

SEQ ID NO: 104 - protein, 16 aa, Rabbit
CARRGATASHRWFTIW

SEQ ID NO: 105 - protein, 16 aa, Rabbit
CGSGANIENEFFNAIW

SEQ ID NO: 106 - protein, 16 aa, Rabbit
CARGDRSHDYDYFKIW

SEQ ID NO: 107 - protein, 18 aa, Rabbit
CARSQDSGSHDDFPFNIW

SEQ ID NO: 108 - protein, 15 aa, Rabbit
CARSPGGIGDAFDPW

SEQ ID NO: 109 - protein, 11 aa, Rabbit
CARGWVGINIW

SEQ ID NO: 110 - protein, 15 aa, Rabbit
CARRADSYGYAYDIW

SEQ ID NO: 111 - protein, 14 aa, Rabbit
CARYGASVTYFNIW

SEQ ID NO: 112 - protein, 17 aa, Rabbit
CARFRILVIVLVPLDLW

SEQ ID NO: 113 - protein, 17 aa, Rabbit
CARGATMTMVRGWLDLW

SEQ ID NO: 114 - protein, 13 aa, Rabbit
CARLGLVVVINIW

SEQ ID NO: 115 - DNA, 405 bp, Rabbit
atggacacgagggcccccactcagctgctggggctcctgctgctctggctcccaggtgccacatttgctcaactgctgacccagactgcatcgcccgtgtctacagctgtgggaggcacagtcaccatcaagtgccagtccagtcagagtgtttttaagaggaagtccttatcctggtatcagcagaaaccagggcaggctcccaaactcctgatctacgatgcatccactctggcatctggggtcccatcacggttcagtggcagtggatctgggacacagttcactctcaccatcagcggcgtgcagtgtgacgatgctgccacttactactgtctaggcagttttgattgtactagtgctgattgtcatgttttcggcggagggaccgaggtggtggtcaaa

SEQ ID NO: 116 - protein, 134 aa, Rabbit
MDTRAPTQLLGLLLLWLPGATFAQLLTQTASPVSTAVGGTVTIKCQSSQSVFKRKSLSWYQQKPGQAPKLLIYDASTLASVPSRFSGSGSGTQFTLTISGVQCDDAATYYCLGSFDCTSADCHVFGGGTEVVVK

SEQ ID NO: 117 - DNA, 39 bp, Artificial Sequence
  Oligonucleotide
aacatgacaatcagcactagtacaatcaaaactgcctag

SEQ ID NO: 118 - DNA, 20 bp, Artificial Sequence
  Oligonucleotide
gctgctctggctcccaggtg

SEQ ID NO: 119 - protein, 112 aa, Rabbit
QLLTQTASPVSTAVGGTVTIKCQSSQSVFKRKSLSWYQQKPGQAPKLLIYDASTLASGVPSRFSGSGSGTQFTLTISGVQCDDAATYYCLGSFDCTSADCHVFGGGTEVVVK

SEQ ID NO: 120 - DNA, 396 bp, Rabbit
atggacacgagggcccccactcagctgctggggctcctgctgctctggctcccaggtgccatatgtgaccctgtgctgacccagactccatcctccgtgtctgcagctgtgggaggcacagtcaccatcaattgccagtccagtcagagggtttggaagaacagctacttatcctggtttcagcagaaaccagggcagcctcccaagcgcctgatctattatacatccactctgccatctggggtcccatcgcggttcaaaggcagtggatctgggacacagttcactctcaccatcagcgacctggagtgtgacgatgctgccacttactactgtctagggagttatagtgatgatatatattctttcggcggagggaccgaggtggtggtcaaa

SEQ ID NO: 121 - protein, 132 aa, Rabbit
MDTRAPTQLLGLLLLWLPGAICDPVLTQTPSSVSAAVGGTVTINCQSSQRVWKNSYLSWFQQKPGQPPKRLIYYTSTLPSGVPSRFKGSGSGTQFTLTISDLECDDAATYYCLGSYSDDIYSFGGGTEVVVK

SEQ ID NO: 122 - DNA, 30 bp, Artificial Sequence
  Oligonucleotide
agaatatatatcatcactataactccctag

SEQ ID NO: 123 - protein, 110 aa, Rabbit
DPVLTQTPSSVSAAVGGTVTINCQSSQRVWKNSYLSWFQQKPGQPPKRLIYYTSTLPSGVPSRFKGSGSGTQFTLTISDLECDDAATYYCLGSYSDDIYSFGGGTEVVVK

SEQ ID NO: 124 - DNA, 402 bp, Rabbit
atggacacgagggcccccactcagctgctggggctcctgctgctctggctcccaggtgccacatttgcgcaagtgctgacccagactgcatcgcccgtgtctgcacctgtgggaggcacagtcaccatcaattgccagtccagtcagagtgtttataataacaacgaattatcttggtatcagcagaaaccaggacagcctcccaagctcctgatctatgctgcatccattttggcatctggggtcccattgcggttcaaaggcagtggatctgggacacagttcactctcaccatcagcgacctggagtgtgacgatgctgccacttactactgtcaaggcagttattatagtggtggttggtacaatgctttcggcggagggaccgaggtggtggtcaaa

SEQ ID NO: 125 - protein, 134 aa, Rabbit
MDTRAPTQLLGLLLLWLPGATFAQVLTQTASPVSAPVGGTVTINCQSSQSVYNNNELSWYQQKPGQPPKLLIYAASILASGVPLRFKGSGSGTQFTLTISDLECDDAATYYCQGSYYSGGWYNAFGGGTEVVVK

SEQ ID NO: 126 - DNA, 36 bp, Artificial Sequence
  Oligonucleotide
agcattgtaccaaccaccactataataactgccttg

SEQ ID NO: 127 - protein, 111 aa, Rabbit
QVLTQTASPVSAPVGGTVTINCQSSQSVYNNNELSWYQQKPGQPPKLLIYAASILASGVPLRFKGSGSGTQFTLTISDLECDDAATYYCQGSYYSGGWYNAFGGGTEVVVK

SEQ ID NO: 128 - DNA, 396 bp, Rabbit
atggacacgagggcccccactcagctgctggggctcctgctgctctggctcccaggtgccacatttgctcaagtgctgacccagactccaccctccgtgtctgcagctgtgggaggcacagtcaccatcagttgccagtccagtcagagcgtttataataataactggttaggctggtatcagcagaaatcagggcagcctcccaagctcctgatctattatgcatccactctggcatctggggtctcatcgcggttcaaaggcagtggatctgggacacagttcactctcaccatcagcgacctggagtgtgacgatgctgccacttattattgtgcaggcggttatagtgatatgatgaatgctttcggcggagggactgaggtggtggttaaa

SEQ ID NO: 129 - protein, 132 aa, Rabbit
MDTRAPTQLLGLLLLWLPGATFAQVLTQTPPSVSAAVGGTVTISCQSSQSVYNNNWLGWYQQKSGQPPKLLIYYASTLASGVSSRFKGSGSGTQFTLTISDLECDDAATYYCAGGYSDMMNAFGGGTEVVVK

SEQ ID NO: 130 - DNA, 30 bp, Artificial Sequence
  Oligonucleotide
gcaggcggttatagtgatatgatgaatgct

SEQ ID NO: 131 - protein, 109 aa, Rabbit
QVLTQTPPSVSAAVGGTVTISCQSSQSVYNNNWLGWYQQKSGQPPKLLIYYASTLASGVSSRFKGSGSGTQFTLTISDLECDDAATYYCAGGYSDMMNAFGGGTEVVVK

SEQ ID NO: 132 - DNA, 384 bp, Rabbit
atggagactgggctgcgctggcttctcctggtcgctgtgctcaaaggtgtccagtgtcagtcggtggaggagtccgggggtcgcctggtcacgcctgggacacccctgacactcgcctgcaccgtctctggattctccctgaggagctatgcaatgatctgggtccgccaggctccaggggaggggctggaatggatcgcggcctttggtactagtggcactacaaactacgcgagctgggcaaaaggccgattcaccatctccagaacctcgaacacggtgtatctcaaaatcaccagtccgacaaccgaggacacggccacctatttctgtgccagacaatggagtttgtggggcccaggcaccctggtcaccgtctcctca

SEQ ID NO: 133 - protein, 128 aa, Rabbit
METGLRWLLLVAVLKGVQCQSVEESGGRLVTPGTPLTLACTVSGFSLRSYAMIWVRQAPGEGLEWIAAFGTSGTTNYASWAKGRFTISRTSNTVYLKITSPTTEDTATYFCARQWSLWGPGTLVTVSS

SEQ ID NO: 134 - DNA, 45 bp, Artificial Sequence
  Oligonucleotide
tgaggagacggtgaccagggtgcctgggccccacaaactccattg

SEQ ID NO: 135 - DNA, 21 bp, Artificial Sequence
  Oligonucleotide
ctgcgctggcttctcctggtc

SEQ ID NO: 136 - protein, 109 aa, Rabbit
QSVEESGGRLVTPGTPLTLTCTVSGFSLSTYAMIWVRQAPGEGLEWIAAFGTSGTTNYATWAKGRFTISRTSNTMDLRITSPTTEDTATYFCARQWSLWGPGTLVTVSS

SEQ ID NO: 137 - DNA, 399 bp, Rabbit
atggagactgggctgcgctggcttctcctggtcgctgtgctcaaaggtgtccagtgtcagtcgctggaggagtccgggggtcgcctggtaacgcctggaggatccctgacactcacctgcacagtctctggaatcgacctcagtacctatccaatgggctgggtccgccaggctccagggaaggggctggaatacatcggaatcgtttttcctagtcttggctcatattacgcgagctgggcaaaaggccgattcaccatctccaaaacctcgtcaaccacggtggatctgcgcatgaccagtctgacaaccgaggacacggccacctatttctgtgccagaggggtaactaatagttgggatccctggggcccaggcaccctggtcaccgtctcctca

SEQ ID NO: 138 - protein, 133 aa, Rabbit
METGLRWLLLVAVLKGVQCQSLEESGGRLVTPGGSLTLTCTVSGIDLSTYPMGWVRQAPGKGLEYIGIVFPSLGSYYASWAKGRFTISKTSSTTVDLRMTSLTTEDTATYFCARGVTNSWDPWGPGTLVTVSS

SEQ ID NO: 139 - DNA, 26 bp, Artificial Sequence
  Oligonucleotide
cccagggatcccaactattagttacc

SEQ ID NO: 140 - protein, 114 aa, Rabbit
QSLEESGGRLVSPGGSLTLTCTVSGIDLSTYPMGWVRQAPGKGLEYIGIVFPSLGSYYASWAKGRFTISKTSSTTVDLRMTSLTAEDTATYFCARGVTNSWDPWGPGTVVTVSS

SEQ ID NO: 141 - DNA, 393 bp, Rabbit
atggagactgggctgcgctggcttctcctggtcgctgtgctcaaaggtgtccagtgtcagtcgctggaggagtccgggggtcgcctggtaacgcctggaggatccctgacactcacctgcacagtctctggaatcgacctcagtagctatggaatgggctgggtccgccaggctccagggaagggtctggaatacatcgcaatcattagttatggtggtagagcatactacgcgagctgggcgaaaggccgattcaccatctccagaacttcgaccacggtggatctgaaaatgaccagtctgacaaccgaggacacggccacctatttctgtgccagaggatttagcgcctttaacttgtggggcccaggcaccctggtcaccgtctcctca

SEQ ID NO: 142 - protein, 131 aa, Rabbit
METGLRWLLLVAVLKGVQCQSLEESGGRLVTPGGSLTLTCTVSGIDLSSYGMGWVRQAPGKGLEYIAIISYGGRAYYASWAKGRFTISRTSTTVDLKMTSLTTEDTATYFCARGFSAFNLWGPGTLVTVSS

SEQ ID NO: 143 - DNA, 26 bp, Artificial Sequence
  Oligonucleotide
gccccacaagttaaaggcgctaaatc

SEQ ID NO: 144 - protein, 112 aa, Rabbit
QSLEESGGRLVTPGGSLTLTCTVSGIDLSSYGMGWVRQAPGKGLEYIAIISYGGRAYYASWAKGRFTISRTSTTVDLKMTSLTTEDTATYFCARGFSAFNLWGPGTLVTVSS

SEQ ID NO: 145 - DNA, 399 bp, Rabbit
atggagactgggctgcgctggcttctcctggtcgctgtgctcaaaggtgtccagtgtcagtcggtggtggaggagtccgggggtcgcctggtcacgcctgggacacccctgacactcacctgcacagcctctggattctccctcagtaggtttgcaatgaggtgggtccgccaggctccagggaaggggctggaatacatcggagccatcgagactgatggtaggacatactacgcgaggtgggcgaaaggccgattcaccatttccaagacctcgaccgcggtgcatctgaagttcaccagtccgacaaccgaggacacgggcacgtatttctgtaccagagggctggttacaatttctactttgtggggcccaggcaccctggtcaccgtctcctca

SEQ ID NO: 146 - protein, 133 aa, Rabbit
METGLRWLLLVAVLKGVQCQSVVEESGGRLVTPGTPLTLTCTASGFSLSRFAMRWVRQAPGKGLEYIGAIETDGRTYYARWAKGRFTISKTSTAVHLKFTSPTTEDTGTYFCTRGLVTISTLWGPGTLVTVSS

SEQ ID NO: 147 - DNA, 28 bp, Artificial Sequence
  Oligonucleotide
gccccacaaagtagaaattgtaaccagc

SEQ ID NO: 148 - protein, 114 aa, Rabbit
QSVVEESGGRLVTPGTPLTLTCTASGFSLSRFAMRWVRQAPGKGLEYIGAIETDGRTYYARWAKGRFTISKTSTAVHLKFTSPTTEDTGTYFCTRGLVTISTLWGPGTLVTVSS

SEQ ID NO: 149 - DNA, 43 bp, Artificial Sequence
  Oligonucleotide
caccatggagactgggctgcgctggcttctcctggtcgctgtg

SEQ ID NO: 150 - DNA, 37 bp, Artificial Sequence
  Oligonucleotide
ctcccgctctccgggtaaatgagcgctgtgccggcga

SEQ ID NO: 151 - DNA, 36 bp, Artificial Sequence
  Oligonucleotide
caggcaggacccagcatggacacgagggcccccact

SEQ ID NO: 152 - DNA, 34 bp, Artificial Sequence
  Oligonucleotide
tcaataggggtgactgttagagcgagacgcctgc

SEQ ID NO: 153 - DNA, 20 bp, Artificial Sequence
  Oligonucleotide
aattcacattgattattgag

SEQ ID NO: 154 - DNA, 40 bp, Artificial Sequence
  Oligonucleotide
cagcgcagcccagtctccatcccgtaagcagtgggttctc

SEQ ID NO: 155 - DNA, 40 bp, Artificial Sequence
  Oligonucleotide
ccgggtaaatgagcgctgtggtttaaacccgctgatcagc

SEQ ID NO: 156 - DNA, 20 bp, Artificial Sequence
  Oligonucleotide
aagccatagagccgaccgca

SEQ ID NO: 157 - DNA, 11 bp, Artificial Sequence
  Oligonucleotide
cggaacaaggg

SEQ ID NO: 158 - DNA, 11 bp, Artificial Sequence
  Oligonucleotide
cggaacaaggg

SEQ ID NO: 159 - protein, 112 aa, Rabbit
QVLTQTASPVSAAVGGTVTIKCQSSQSVYKRKSLSWYQQKPGQAPKLLIYDASTLASGVPSRFSGSGSGTQFTLTISGVQCDDAATYYCLGSFDCTSADCHVFGGGTEVVVK

SEQ ID NO: 160 - protein, 112 aa, Rabbit
QVLTQTASPVSAAVGGTVTINCQSSQSVYKRKSLSWYQQKPGQAPKLLIYDASTLASGVPSRFKGSGSGTQFTLTISGVQCDDAATYYCLGSFDCTRADCHVFGGGTEVVVK

SEQ ID NO: 161 - protein, 112 aa, Rabbit
QVLTQTASPVSAAVGGTVTIKCQSSQSVYKRKSLSWYQQKPGQAPKLLIYDASTLASGVPSRFKGSGSGTQFTLTISGVQCDDAATYYCLGSFDCTRADCHVFGGGTEVVVK

SEQ ID NO: 162 - protein, 112 aa, Rabbit
QVLTQTASPVSAAVGGTVTIKCQSSQSVYKRKSLSWYQQKPGQPPKLLIYDASTLASGVPSRFKGSGSGTQFTLTISGVQCDDAATYYCLGSFDCTSADCHVFGGGTEVVVK

SEQ ID NO: 163 - protein, 112 aa, Rabbit; identical to SEQ ID NO: 162

SEQ ID NO: 164 - protein, 112 aa, Rabbit
QVLTQTASPVSAAVGGTVTIKCQSSQSVYKSKHCSWYQQKPGQPPKLLIYDASTLASGVPSRFKGSGSGTEFTLTISGVQCDDAATYYCLGSYDCSTADCHVFGGGTGVVVK

SEQ ID NO: 165 - protein, 112 aa, Rabbit
QVLTQTPSSVSAAVGGTVTIKCQSSQSVYKSKHLSWFQQKPGQAPKLLIYDTSTLASGVPSRFSGSGSGTQFTLTISGVQCDDAAAYYCLGSYDCLSADCHVFGGGTEVVVK

SEQ ID NO: 166 - protein, 112 aa, Rabbit; identical to SEQ ID NO: 165

SEQ ID NO: 167 - protein, 112 aa, Rabbit
QVLTQTPSSVSAAVGGTVTIKCQSSQSVYKSKHLSWFQQKPGQAPKLLIYDTSTLASGVPSRFKGSGSGTQFTLTISGVQCDDAAAYYCLGSYDCLSADCHVFGGGTEVVVK

SEQ ID NO: 168 - protein, 112 aa, Rabbit
QVLTQTPSPVSAAVGGTVTINCQASQSVYKSKHLSWYQQKPGQPPKLLIYDASKLASGVPSRFKGSGSGTHFTLTISGVQCDDAATYYCLGSYDCSRADCHVFGGGTEVVVK

SEQ ID NO: 169 - protein, 112 aa, Rabbit
QVLTQTPSPVSAAVGGTVTINCRASQTVYKSKHLSWYQQKPGQPPKLLIYDASKLASGVPSRFKGSGSGTQFTLTISGVECDDAATYYCLGSYDCSSTDCHVFGGGTEVVVK

SEQ ID NO: 170 - protein, 112 aa, Rabbit
QVLTQTPSPVSAAVGGTVTINCQASQRVYKNKHLSWYQQKPGQRPKLLIYDASTLASGVPSRFKGSGSGTQFTLTLSGVQCDDAATYYCLGSYDCTITDCHVFGGGTEVVVK

SEQ ID NO: 171 - protein, 112 aa, Rabbit
QVLTQTPSPVSAAVGGTVTINCQASQSVYKRKYLSWYQKKPGQAPKLLIYDASTLASGVPSRFSGSGSGTQFTLTISGVQCDDAATYYCLGSYDCSRTDCHVFAGGTELVVK

SEQ ID NO: 172 - protein, 110 aa, Rabbit
DPVLTQTPSSVSAAVGGTVTINCQSSQSVYKNKYLSWFQQKPGQPPKRLIYYVSTLASGVPSRFKGSGSGTQFTLTISGVQCDDAATYYCLGSYSNDIYSFGGGTEVVVK

SEQ ID NO: 173 - protein, 109 aa, Rabbit
QSVEEFGGRLVTPGTPLTLTCTVSGFSLSTVAMIWVRQAPGEGLEWIAAFGTSGTTNYATWAKGRFTISRTSNTVDLRITRPTTEDTATYFCARQWSLWGPGTLVTVSS

SEQ ID NO: 174 - protein, 109 aa, Rabbit
QSVEESGGRLVTPGTPLTLTCTVSGFSLSTVAMIWVRQAPGEGLEWIAAFGTSGTTNYATWAKGRFTISRTSNTVDLRITRPTTEDTATYFCARQWSLWGPGTLVTVSS

SEQ ID NO: 175 - protein, 109 aa, Rabbit
QSVEESGGRLVTPGTPLTLTCTASGFSLSTVAMIWVRQAPGEGLQWIAAFGTRGTTNYATWAKGRFTISRTSNTVDLRITSPTTEDTATYFCARQWSLWGPGTLVTVSS

SEQ ID NO: 176 - protein, 109 aa, Rabbit
QSVEESGGRLVTPGTPLTLTCTVSGVSLRGYAMIWVRQAPGEGLKWIAAFGTSGTTNYASWAKGRFTISRTSNTVDLKITSPTTEDTATYFCARQWSLWGPGTLVTVSS

SEQ ID NO: 177 - protein, 109 aa, Rabbit
QSVEESGGRLVTPGTPLTLTCTISGVSLRGYAMIWVRQAPGEGLKWIAAFGTSGTTNYASWAKGRFTISRTSNTVDLKITSPTTEDTATYFCARQWSLWGPGTLVTVSS

SEQ ID NO: 178 - protein, 109 aa, Rabbit
QSVEESGGRLVTPGTPLTLTCTVSGFSLRSYAMIWVRQAPGEGLEWIAAFGTSGTTNYASWAKGRFTISRTSNTVYLKITSPTTEDTATYFCARQWSLWGPGTLVTVSS

SEQ ID NO: 179 - protein, 109 aa, Rabbit
QSVEESGGRLVTPGTPLTLTCTVSGFSLRSYAMIWVRQAPGEGLEWIAAFGTSGTTNYASWAKGRFTISRTSNTVYLRITSPTTEDTATYFCARQWSLWGPGTLVTVSS

SEQ ID NO: 180 - protein, 109 aa, Rabbit
QSVEESGGRLVTPGTPLTLTCTVSGFSLSSYAMIWVRQAPGEGLEWIAAFGTSGSTNYASWAKGRFTISRTSNTMHLKITSPTTEDTATYFCARQWSLWGPGTLVTVSS

SEQ ID NO: 181 - protein, 109 aa, Rabbit
QSVEESGGRLVTPGTPLTLTCTVSGIDLSNYAMIWVRQAPGEGLEWIAAFGTSGSASYASWAKGRFTISKTSNTVDLKITSPTTEDTATYFCARQWSLWGPGTLVTVSS

SEQ ID NO: 182 - protein, 109 aa, Rabbit
QSVEESGGRLVTPGTPLTLTCTVSGIDLSKYAMIWVRQAPGEGLKWIAALGASGTTNYASWAKGRFTISRTSNTVDLKITSPTTEDTATYFCARQWSLWGPGTLVTVSS

SEQ ID NO: 183 - protein, 109 aa, Rabbit
QSVEESGGRLVTPGTPLTLTCTVSGFSLSSYAMIWVRQAPGKGLEWIAAFGTSGTTNYASWAKGRFTISKTSNTVDLKITSPTPGDTATYFCARQWSLWGPGTLVTISS

SEQ ID NO: 184 - protein, 109 aa, Rabbit
QSVEESGGRLVTPGTPLTLTCTVSGFSLSSYAMIWVRQAPGKGLEWIAAFGTSGTRNYASWAKGRFTISRTSTTVDLKMTSLTTEDTATYFCARQWSLWGPGTLVTVSS

SEQ ID NO: 185 - protein, 109 aa, Rabbit
QSVEESGGRLVTPGTPLTLTCTVSGFSLGSYAMIWVRQAPGKGLEWIAAFGTSGTRNYASWAKGRFTISRTSTTVDLKMTSLTTEDTATYFCARQWSLWGPGTLVTVSS

SEQ ID NO: 186 - protein, 114 aa, Rabbit
QSLEESGGRLVTPGGSLTLTCTVSGIDLSTYPMGWVRQAPGKGLEYIGIVFPSLGSYYASWAKGRFTISKTSSTTVDLRMTSLTTEDTATYFCARGVTNSWDPWGPGTLVTVSS

SEQ ID NO: 187 - protein, 114 aa, Rabbit
QSLEESGGRLVTPGGSLTLTCTVSGIDLSTYPMGWVRQAPGKGLEYIGIVFPNIGSYYASWAKGRFTISSTSSTTVDLRMTSLTTEDTATYFCARGVTNSWDPWGPGTLVTVSS

(19) United States
(12) Patent Application Publication
(10) Pub. No.: US 2014/0162904 A1
(43) Pub. Date: Jun. 12, 2014

(54) Library of Cells That Produce Antibodies That Comprise Non-Naturally Paired IGH and IGL Variable Domains

(71) Applicant: Abcam PLC (EPITOMICS, Inc.), Cambridge (GB)

(72) Inventor: GUO-LIANG YU, Hillsborough, CA (US)

(21) Appl. No.: 14/157,334

(22) Filed: Jan. 16, 2014

Related U.S. Application Data
(63) Continuation of application No. 12/483,956, filed on Jun. 12, 2009.

(51) Int. Cl. C07K 16/46

(52) U.S. Cl. CPC C07K 16/46
USPC 506/14

(57) ABSTRACT

A method for producing a library of engineered-antibody producing cells is provided. In certain cases, the method includes isolating nucleic acid sequences encoding IgH variable regions and IgL variable regions from a plurality of antibody producing cells, and introducing the nucleic acids into host cells to obtain cells that produce antibodies comprising non-naturally paired IgH and IgL variable chains.

### INTRODUCTION

Antibodies are proteins that bind a specific antigen. Generally, antibodies are specific for their targets, have the ability to mediate immune effector mechanisms, and have a long half-life in serum. Such properties make antibodies powerful therapeutics. Monoclonal antibodies are used therapeutically for the treatment of a variety of conditions including cancer, inflammation, and cardiovascular disease. There are currently over ten therapeutic antibody products on the market and hundreds in development.

There is a constant need for new antibodies and methods for making the same.

### SUMMARY

A method of producing a library of engineered-antibody producing cells is provided. In some cases, the method includes isolating a plurality of antibody producing cells from a first animal immunized with an antigen, wherein the plurality of antibody producing cells express antibodies that bind to the antigen; obtaining from the antibody producing cells a plurality of first nucleic acids encoding the IgH variable regions of the antibodies and a plurality of second nucleic acids encoding the IgL variable region of the antibodies; introducing expression cassette pairs in a plurality of host cells, each expression cassette pair includes: i) a first nucleic acid of said plurality of first nucleic acids; and ii) a second nucleic acid of said plurality of second nucleic acids, thereby obtaining cells comprising the first and second nucleic acids, wherein the first and second nucleic acids are not paired together in the antibody producing cells to produce the library of engineered-antibody producing cells that produce antibodies comprising non-naturally paired IgH and IgL variable chains. In particular embodiments, the isolating step utilizes binding of the plurality of antibody producing cells to the antigen. In some cases, the first and second nucleic acids of the expression cassette pair are present in a single polynucleotide, while in alternative examples, the first and second nucleic acids of the expression cassette pair are present in a separate first and a second polynucleotide, respectively. In certain examples, the first and second nucleic acids of the expression cassette pair are systematically paired. In alternative embodiments, the first and second nucleic acids of the expression cassette pair are randomly paired. In particular embodiments, the animal is a rabbit. In certain cases, the plurality of antibody producing cells comprises at least ten antibody producing cells. In exemplary cases, the plurality of antibody producing cells include cells that are related by lineage to a precursor B-cell. In some cases, the plurality of host cells are mammalian host cells. In some examples, the library of engineered-antibody producing cells contain cells that produce antibodies that bind to the antigen.

Also provided is a library of engineered-antibody producing cells and a method of screening the same.

### BRIEF DESCRIPTION OF THE DRAWINGS

FIG. 1 is a flow diagram illustrating one embodiment of the invention.

FIG. 2 shows the amino acid sequences of selected KDR-binding antibodies. Page 1 of FIG. 2 shows amino acid sequences of the heavy chains. Page 2 of FIG. 2 shows amino acid sequences of the corresponding light chains. The amino acid sequences shown in FIG. 2 are of antibodies that specifically bind to KDR and block VEGF activity. From top to bottom, FIG. 2 (page 1 of 2) SEQ ID NOS: 1-47 and FIG. 2 (page 2 of 2) SEQ ID NOS: 48-94.

FIG. 3 shows the amino acid sequence of 20 exemplary VH3 regions of unrelated rabbit antibodies. From top to bottom SEQ ID NOS: 95-114.

FIGS. 4A-4H show exemplary methods by which related antibodies can be amplified.

### DEFINITIONS

Before the present subject invention is described further, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an antibody” includes a plurality of such antibodies and reference to “a framework region” includes reference to one or more framework regions and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The term “nucleic acid” encompasses DNA, RNA, single stranded or double stranded and chemical modifications thereof. The terms “nucleic acid” and “polynucleotide” are used interchangeably herein.

The term “expression”, as used herein, refers to the process by which a polypeptide is produced based on the nucleic acid sequence of a gene. The process includes both transcription and translation.

The term “expression cassette” refers to a nucleic acid construct capable of directing the expression of a gene/coding sequence of interest, which is operably linked to a promoter of the expression cassette. Such cassettes can be a linear nucleic acid or can be present in a “vector”, “vector construct”, “expression vector”, or “gene transfer vector”, in order to transfer the expression cassette into target cells. Thus, the term includes cloning and expression vehicles, as well as viral vectors.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The term “plurality” refers to more than 1, for example more than 2, more than about 5, more than about 10, more than about 20, more than about 50, more than about 100, more than about 200, more than about 500, more than about 1000, more than about 2000, more than about 5000, more than about 10,000, more than about 20,000, more than about 50,000, more than about 100,000, usually no more than about 200,000. A “population” contains a plurality of items.

The term “introduced” in the context of inserting a nucleic acid sequence into a cell, means “transfection”, or ‘transformation”, or “transduction” and includes reference to the incorporation of a nucleic acid sequence into a eukaryotic or prokaryotic cell wherein the nucleic acid sequence may be present in the cell transiently or may be incorporated into the genome of the cell (e.g., chromosome, plasmid, plastid, or mitochondrial DNA), converted into an autonomous replicon.

The terms “antibody” and “immunoglobulin” are used interchangeably herein. These terms are well understood by those in the field, and refer to a protein consisting of one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)₂, and or other antibody fragments that retain specific binding to antigen, and monoclonal antibodies.

Antibodies may exist in a variety of other forms including, for example, Fv, Fab, and (Fab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed., 1984, and Hunkapiller and Hood, Nature, 323, 15-16, 1986).

An immunoglobulin light or heavy chain variable region consists of a framework region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

The term “chimeric antibodies” refer to antibodies whose light and heavy chain genes have been constructed, typically by genetic engineering, from antibody variable and constant region genes belonging to different species. For example, the variable segments of the genes from a mouse monoclonal antibody may be joined to human constant segments, such as gamma 1 and gamma 3. An example of a therapeutic chimeric antibody is a hybrid protein composed of the variable or antigen-binding domain from a rabbit antibody and the constant or effector domain from a human antibody, although other mammalian species may be used.

The term “humanized antibody” or “humanized immunoglobulin” refers to an non-human (e.g., mouse or rabbit) antibody containing one or more amino acids (in a framework region, a constant region or a CDR, for example) that have been substituted with a correspondingly positioned amino acid from a human antibody. In general, humanized antibodies produce a reduced immune response in a human host, as compared to a non-humanized version of the same antibody.

The terms “polypeptide” and “protein”, used interchangeably herein, refer to a polymeric form of amino acids of any length, which can include coded and non-coded amino acids, chemically or biochemically modified or derivatized amino acids, and polypeptides having modified peptide backbones.

The term “natural” antibody refers to an antibody in which the heavy and light immunoglobulins of the antibody have been naturally paired by the immune system of a multi-cellular organism. Spleen, lymph nodes and bone marrow are examples of tissues that produce natural antibodies. For example, the antibodies produced by the antibody producing cells isolated from a first animal immunized with an antigen are natural antibodies.

The term “non-naturally paired”, with respect to VH and VL chains of an engineered antibody generated by the subject method, refers to a VH and VL pair that is not found in a natural antibody. Thus, a non-naturally paired antibody is a combination of VH and VL chain of two different natural antibodies. However, the VH and VL chains of a non-naturally paired antibody are not mutated relative to the VH and VL chains of the two different antibodies which provided the VH and VL chains. For example, these “non-naturally paired” IgH and IgL chains of the engineered antibody may contain the IgH variable chain from a first antibody producing cell obtained from an animal and the IgL variable chain of second antibody producing cell obtained from the same animal, where the amino acid sequence of the antibody produced by the first cell is different from the amino acid sequence of the antibody produced by the first cell. The engineered antibody comprised of “non-naturally paired” IgH and IgL chains are not made by phage display. As such, the subject engineered antibodies do not usually contain any viral (e.g., bacteriophage M13)-derived sequences.

The term “related antibody producing cells” or “antibody producing cells related by lineage”, as will be described in greater detail below, are antibody producing cells that have a common B cell ancestor and produce antibodies with a similar sequence. Such a B cell ancestor contains a genome having a rearranged light chain VJC region and a rearranged heavy chain VDJC region, and produces an antibody that has not yet undergone affinity maturation. “Naïve” or “virgin” B cells present in spleen tissue, are exemplary B cell common ancestors. Related antibodies produced by related antibody producing cells bind to the same epitope of an antigen and are typically very similar in sequence, particularly in their L3 and H3CDRs. Both the H3 and L3 CDRs of related antibodies have an identical length and a near identical sequence (i.e., differ by up to 5, i.e., 0, 1, 2, 3, 4 or 5 residues). Related antibodies are related via a common antibody ancestor, the antibody produced in the naïve B cell ancestor. The term “related antibodies” is not intended to describe a group of antibodies that are not produced by cells that arise from the same ancestor B-cell.

The term “plurality” refers to at least 2, more than about 5, more than about 10, more than about 50, more than about 100, more than about 200, more than about 500, more than about 1000, more than about 2000, more than about 5000, or more than about 10,000 or more than about 50,000 or more.

The terms “treating” or “treatment” of a condition or disease refer to providing a clinical benefit to a subject, and include: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms.

### DETAILED DESCRIPTION OF EXEMPLARY EMBODIMENTS

A method of producing a library of engineered-antibody producing cells is provided. In some cases, the method includes isolating a plurality of antibody producing cells from a first animal immunized with an antigen, wherein the plurality of antibody producing cells express antibodies that bind to the antigen; obtaining from the antibody producing cells a plurality of first nucleic acids encoding the IgH variable regions of the antibodies and a plurality of second nucleic acids encoding the IgL variable region of the antibodies; introducing expression cassette pairs in a plurality of host cells, each expression cassette pair includes: i) a first nucleic acid of said plurality of first nucleic acids; and ii) a second nucleic acid of said plurality of second nucleic acids, thereby obtaining cells comprising the first and second nucleic acids, wherein the first and second nucleic acids are not paired together in the antibody producing cells to produce the library of engineered-antibody producing cells that produce antibodies comprising non-naturally paired IgH and IgL variable chains. In particular embodiments, isolating utilizes binding of the plurality of antibody producing cells to the antigen.

### Method for Producing Library of Engineered-Antibody Producing Cells

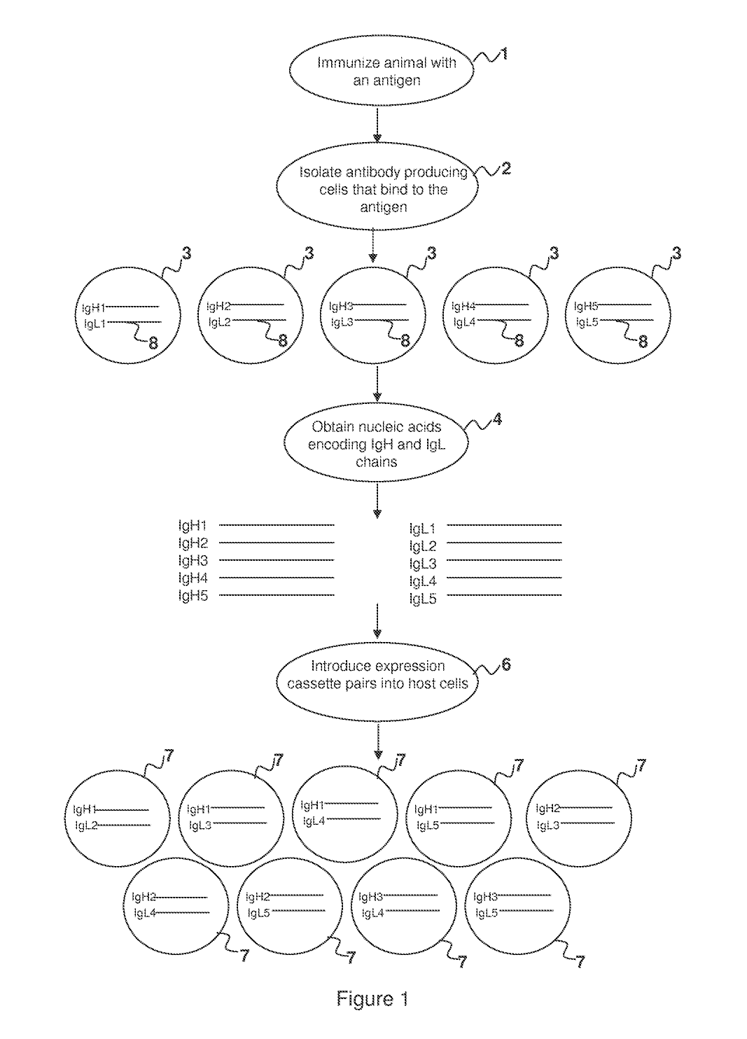

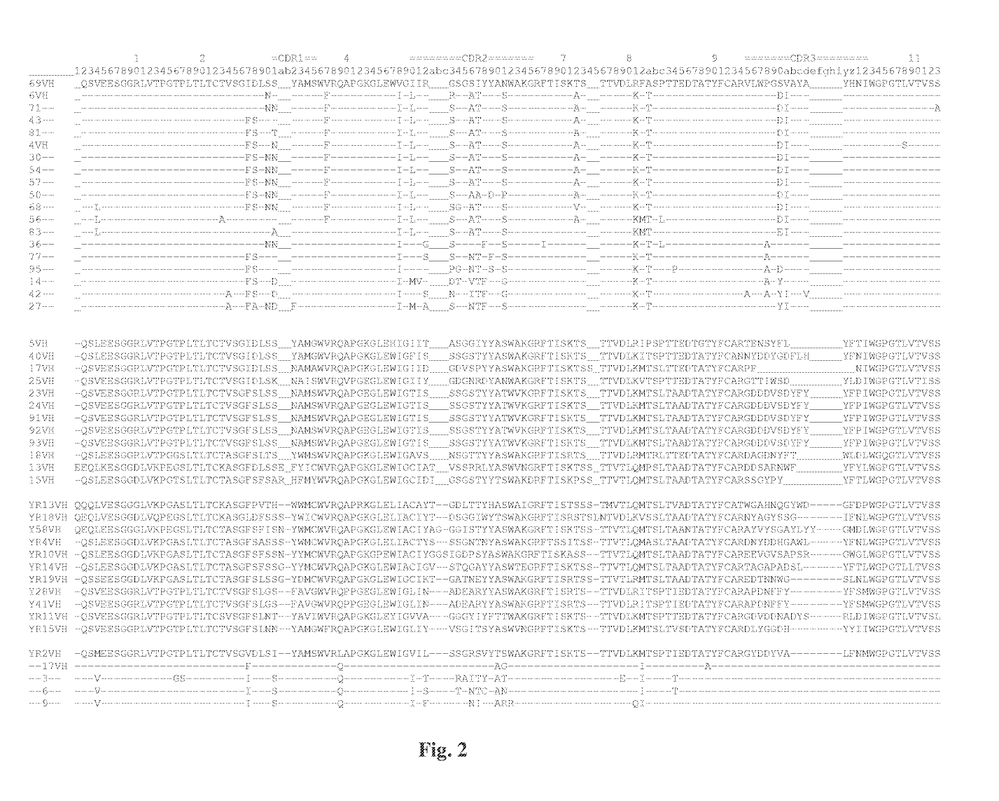

As noted above a method for producing a library of engineered-antibody producing cells is provided. With reference to FIG. 1, the subject method involves immunizing an antibody-producing animal with a selected antigen 1, and isolating from the animal antibody producing cells that bind to the antigen 2. In FIG. 1, five different antibody producing cells 3 are shown, which may be more. Each of these cells 3 produce natural antibodies containing naturally paired IgH and IgL chains 8. The nucleic acids encoding the IgH and IgL chains of these natural antibodies are obtained 4. These nucleic acids are used to produce expression cassette pairs that are introduced into a plurality of host cells 6 to produce a library of engineered antibody producing cells 7. In FIG. 1, nine different engineered antibody producing cells 7 are shown, which may be more. These cells 7 produce engineered antibodies that contain non-naturally paired IgH and IgL chains. Thus, this method achieves acquiring the nucleic acids 8 encoding IgH and IgL chains of natural antibodies that bind to the antigen and the non-natural pairing of these nucleic acids to obtain engineered antibodies that increase the diversity of antibodies.

In general, the method includes isolating a plurality of antibody producing cells from an animal immunized with an antigen, where the plurality of antibody producing cells express antibodies that bind to the antigen. In exemplary embodiments, the antibodies produced by the plurality of antibody producing cells are uncharacterized except for the knowledge that the antibodies bind to the antigen with which the animal was immunized. In other words, the epitope to which these antibodies bind may be unknown, the nucleic acid or amino acid sequence of the variable regions of the IgH and IgL chain of these antibodies may be unknown, etc. In certain cases, the plurality of antibody producing cells may be isolated based on their binding to the antigen.

As will be described in more detail below, a plurality of first nucleic acids encoding the IgH variable regions and a plurality of second nucleic acids encoding the IgL variable regions of the antibodies is obtained from the isolated cells. In certain embodiments, expression cassette pairs may be generated from these pluralities of nucleic acids encoding IgH and IgL variable regions. Generally, an expression cassette pair may include a first nucleic acid of the plurality of first nucleic acids encoding the IgH variable regions and a second nucleic acid of the plurality of second nucleic acids encoding the IgL variable regions. In some cases, the first and second nucleic acids of the expression cassette pair may be present in a single polynucleotide. For example, the first nucleic acid encoding an IgH variable region and the second nucleic acid encoding an IgL variable region may be cloned into a single vector. In embodiments where the first nucleic acid encoding an IgH variable region and the second nucleic acid encoding an IgL variable region are present in a single polynucleotide, the first and second nucleic acid may be randomly paired or systematically paired. Random and systematic pairing is described below. Alternatively, the first and second nucleic acids of the expression cassette pair may be present in separate first and second polynucleotides. For example, the first nucleic acid encoding an IgH variable region and the second nucleic acid encoding an IgL variable region may be cloned into two separate vectors, where the two vectors have different selectable markers. In certain cases, where the first and the second nucleic acids are present in two separate polynucleotides, the first and the second nucleic acids of the expression cassette pair may be randomly paired. Alternatively, the first and the second nucleic acids of the expression cassette pair may be systematically paired. The expression cassette pairs may be introduced into a plurality of host cells to obtain cells that may contain a first nucleic acid encoding an IgH variable region and a second nucleic acid encoding an IgL variable region that are not found together in the antibody producing cells to produce the library of engineered-antibody producing cells that produce antibodies with non-naturally paired IgH and IgL variable chains. In other words, the library may contain cells that produce antibodies with a combination of IgH and IgL chains that is not found amongst the antibody producing cells from which the nucleic acids encoding IgH and IgL chains were obtained. Said another way, the library may contain cells that produce engineered-antibodies. These engineered-antibodies combine IgH and IgL variable regions from two different antibody producing cells, where the antibodies produced from the two different antibody producing cells bind to the same antigen but are non-identical in amino acid sequence.

### Antibody Producing Cells

An antibody-producing cell is a cell that produces antibodies. Such cells are typically cells involved in a mammalian immune response, such as a B-lymphocyte or its progeny including the plasma cell, and produce immunoglobulin heavy and light chains that have been “naturally paired” by the immune system of the host. These cells may either secrete antibodies (antibody-secreting cells) or maintain antibodies on the surface of the cell without secretion into the cellular environment.

An antibody-producing cell may be obtained from an animal which has not been immunized with a selected antigen, which has been immunized with a selected antigen, or which has developed an immune response to an antigen as a result of disease or condition. Animals may be immunized with a selected antigen using any of the techniques well known in the art suitable for generating an immune response (see Handbook of Experimental Immunology D. M. Weir (ed.), Vol 4, Blackwell Scientific Publishers, Oxford, England, 1986). The term “selected antigen” includes any substance to which an antibody may be made, including, among others, proteins, carbohydrates, inorganic or organic molecules, transition state analogs that resemble intermediates in an enzymatic process, nucleic acids, cells, including cancer cells, cell extracts, pathogens, including living or attenuated viruses, bacteria and the like. As will be appreciated by one of ordinary skill in the art, antigens, especially antigen which are of low immunogenicity, may be accompanied with an adjuvant or hapten in order to increase the immune response (for example, complete or incomplete Freund's adjuvant) or with a carrier such as keyhole limpet hemocyanin (KLH). Suitable antigens include extracellularly-exposed fragments of Her2, GD2, EGF-R, CEA, CD52, CD20, Lym-1, CD6, complement activating receptor (CAR), EGP40, VEGF, tumor-associated glycoprotein TAG-72 AFP (alpha-fetoprotein), BLyS (TNF and APOL-related ligand), CA125 (carcinoma antigen 125), CEA (carcinoembrionic antigen), CD2 (T-cell surface antigen), CD3 (heteromultimer associated with the TCR), CD4, CD11a (integrin alpha-L), CD14 (monocyte differentiation antigen), CD20, CD22 (B-cell receptor), CD23 (low affinity IgE receptor), CD25 (IL-2 receptor alpha chain), CD30 (cytokine receptor), CD33 (myeloid cell surface antigen), CD40 (tumor necrosis factor receptor), CD44v6 (mediates adhesion of leukocytes), CD52 (CAMPATH-1), CD80 (costimulator for CD28 and CTLA-4), complement component C5, CTLA, EGFR, eotaxin (cytokine A11), HER2/neu, HLA-DR, HLA-DR10, HLA ClassII, IgE, GPiib/iiia (integrin), Integrin aVβ3, Integrins a4β1 and a4β7, Integrin β2, IFN-gamma, IL-1β, IL-4, IL-5, IL-6R (IL6 receptor), IL-12, IL-15, KDR (VEGFR-2), lewisy, mesothelin, MUC1, MUC18, NCAM (neural cell adhesion molecule), oncofetal fibronectin, PDGFβR (Beta platelet-derived growth factor receptor), PMSA, renal carcinoma antigen G250, RSV, E-Selectin, TGFbeta1, TGFbeta2, TNFalpha, TRAIL-R1, VAP-1 (vascular adhesion protein 1) or TNFα, or the like.

In many embodiments, a peptide having the amino acid sequence corresponding to a portion of an extracellular domain of one of the above-listed proteins is employed as an antigen.

Many warm-blooded animals, in particular mammals such as humans, rabbits, mice, rats, sheep, cows, pigs and ayes such as chickens and turkeys, may be used in order to obtain antibody-forming cells. However, rabbits and mice are generally preferred because of their ease in handling, well-defined genetic traits, and the fact that they may be readily sacrificed. Procedures for immunizing animals are well known in the art, and are described in Harlow et al., (Antibodies: A Laboratory Manual, First Edition (1988) Cold Spring Harbor, N.Y.). Antibody-producing cells may also be obtained from a subject which has generated the cells during the course of a selected disease or condition. For instance, antibody-producing cells from a human with a disease of unknown cause, such as rheumatoid arthritis, may be obtained and used in an effort to identify antibodies which have an effect on the disease process or which may lead to identification of an etiological agent or body component that is involved in the cause of the disease. Similarly, antibody-producing cells may be obtained from subjects with disease due to known etiological agents such as malaria or AIDS. These antibody-producing cells may be derived from the blood, lymph nodes or bone marrow, as well as from other diseased or normal tissues. Antibody-producing cells may also be prepared from blood collected with an anticoagulant such as heparin or EDTA. The antibody-producing cells may be further separated from erythrocytes and polymorphs using standard procedures such as centrifugation with Ficoll-Hypaque (Pharmacia, Uppsula, Sweden). Antibody-producing cells may also be prepared from solid tissues such as lymph nodes or tumors by dissociation with enzymes such as collagenase and trypsin in the presence of EDTA.

In certain embodiments, the antibody producing cells are B-cells from a rabbit that has been immunized with an antigen. A proportion of these B-cells may be composed of B-cells that are related by lineage to a precursor B-cell. These lineage-related B-cell arise from the same precursor B-cell but produce antibodies of non-identical sequence as these B-cells have undergone somatic hypermutation. The antibodies produced by lineage-related B-cells have H3 CDRs that are almost identical, as well as L3 CDRs that are almost identical. Thus, antibodies produced by lineage-related B-cells may have L3 and H3 CDRs that are each identical in length and have near identical sequences (i.e., that contain 0, 1, 2, 3, 4 or 5 amino acid changes). In other words, the L3 CDRs of the antibodies produced by lineage-related B-cells may be identical in length and near identical in sequence and the H3 CDRs antibodies produced by lineage-related B-cells may be identical in length and near identical in sequence. Examples of related antibodies and methods for identifying related antibodies are described in U.S. Application Ser. Nos. 61/151,052 and 61/151,397. The methods for identifying related antibodies described in U.S. Application Ser. Nos. 61/151,052 and 61/151,397 are herein incorporated by reference.

Antibody-producing cells may also be obtained by culture techniques such as in vitro immunization. Examples of such methods are described Reading in Methods in Enzymology (21:18-33 J. J. Langone, H. H. van Vunakis (eds.), Academic Press Inc., N.Y.; 1986). Briefly, a source of antibody-producing cells, such as a suspension of spleen or lymph node cells, or peripheral blood mononuclear cells are cultured in medium such as RPMI 1640 with 10% fetal bovine serum and a source of the substance against which it is desired to develop antibodies. This medium may be additionally supplemented with amounts of substances known to enhance antibody-forming cell activation and proliferation such as lipopolysaccharide or its derivatives or other bacterial adjuvants or cytokines such as IL-1, IL-2, IL-4, IL-5, IL-6, GM-CSF, and IFN-gamma. To enhance immunogenicity, the selected antigen may be coupled to the surface of cells, for example, spleen cells, by conventional techniques such as the use of biotin/avidin.

In certain embodiments, the antibody producing cells are a plurality of hybridoma cells which all produce antibodies that bind to the same antigen. The antibodies produced by these hybridoma cells are natural antibodies because the pairing of VH and VL chains of these antibodies is the same as that found in the antibody producing cell that was used to make the hybridoma. In these embodiments, the step of isolating antibody producing cells includes isolating the antibody producing cells and using them to produce hybridomas. The method may involve fusing cells (for example, spleen cells) from an animal with a fusion partner to produce hybridoma cells; screening the hybridoma cells using traditional techniques to identify hybridoma cells that produce antibodies that bind to an antigen. Once identified the hybridoma cells can be employed in the next step of the subject method. In alternative embodiments, the antibody producing cells are not hybridoma cells.

A suitable animal producing antibody producing cells may be identified using a number of assays for detecting presence of such cells in an animal. In some cases, the serum obtained from an animal immunized with an antigen may be assayed using ELISA, for example. Once a suitable animal producing antibodies has been identified or produced, spleen, lymph node or bone marrow tissue is typically removed, and a cell suspension of antibody-producing cells is prepared using techniques well known in the art.

In exemplary embodiments, an affinity purification method is utilized to isolate antibody producing cells. The antigen with which the animal was immunized may be immobilized on a solid phase and used to selectively retain antibody producing cells that express an antibody on their surface that binds to the antigen, while other cells are washed away. The retained cells may then be eluted by a variety of methods, such as, by using an excess of the antigen, chaotropic agents, changing the pH, salt concentration, etc. Any of the well known methods for immobilizing or coupling antigen to a solid phase may be used. For example, when the antigen is a cancer cell, appropriately treated microtiter plate that will bind to cells may be used, such as microtiter plates for cell culture. In the instances where the antigen is a protein, the protein may be covalently attached to a solid phase, for example, sepharose beads, by well known techniques, etc. Alternatively, a labeled antigen may be used to specifically label cells that express an antibody that binds to the antigen and the labeled cells may then be isolated by FACS. Generally, methods for antibody purification may be adapted to isolate antibody producing cells. Such methods are well known and are described in, for example, J Immunol Methods. 2003 November; 282(1-2):45-52; J Chromatogr A. 2007 Aug. 10; 1160(1-2):44-55; J Biochem Biophys Methods. 2002 May 31; 51 (3):217-31. Cells may also be isolated using magnetic beads or by any other affinity solid phase capture method, protocols for which are known.

The plurality of antibody producing cells isolated by the aforementioned methods may be substantially pure, i.e., substantially free of other cells that do not produce an antibody or do not produce an antibody that specifically binds to the antigen. The term “substantially pure”, as used herein refers, to an isolated population of plurality of antibody producing cells, where cells that express antibodies that specifically bind to the antigen make up at least 30%, at least 40%, at least 50%, at least 60%, at least 70% or more of the total population of cells. The isolated population of plurality of antibody producing cells may be used in the next step as a mixture of cells, or alternatively, they may be separated into single cells, e.g., by dilution and deposition into individual wells of a microtiter plate, or may be separated into a pool of approximately ten cells in each well of a microtiter plate.

The plurality of antibody producing cells isolated by the aforementioned methods may comprise at least 5, at least 10, at least 30, at least 60, at least 100, at least 300, at least 500, at least about 1000, at least about 3000, at least about 100,000, or more antibody producing cells.

In other embodiments, antibody-producing cells may be enriched from a single cell suspension prior to isolating antibody-producing cells that produce antibodies that bind to the antigen used to immunize the animal. Antibody-producing cells may be enriched by methods based upon the size or density of the antibody-forming cells relative to other cells. An example of the use of Percoll to separate cells according to density is described by van Mourik and W. P. Zeizlmaker in Methods in Enzymology 121:174-182 (J. J. Langone, H. H. van Vunakis (eds.), Academic Press Inc., N.Y.). Gradients of varying density of solutions of bovine serum albumin can also be used to separate cells according to density. (See N. Moav and T. N. Harris, J. Immunol 105:1512, 1970; see also Raid, D. J. in SELECTED METHODS IN CELLULAR IMMUNOLOGY, B. Mishell and S. Shiigi (eds.), W. H. Freeman and Co., San Francisco, 1987).

Antibody-producing cells may also be enriched and plated using other methods. Exemplary antibody-producing cell enrichment methods include performing flow cytometry (FACS) of cell populations obtained from rabbit spleen, bone marrow, lymph node or other lymph organs, e.g., through incubating the cells with labeled anti-rabbit IgG and sorting the labeled cells using a FACSVantage SE cell sorter (Becton-Dickinson, San Jose, Calif.). In some embodiments, single or nearly single antibody-producing cells are deposited in microtiter plates. If the FACS system is employed, sorted cells may be deposited after enrichment directly into a microtiter plate.

In certain embodiments, the antibody producing cells may secrete the antibody instead of expressing the antibody in the cell surface. In such cases, the antibody producing cells may first be separated into individual wells of a microtiter plate or pooled into sets of approximately five cells per well and the media from these wells may be tested for presence of antibodies that bind to the antigen with which the animal was immunized.

Generally, these antibody producing cells and the antibodies produced by these cells are not well characterized. As such, although the antibody-producing cells are isolated or selected based on the production of antibodies that specifically bind to the antigen, the epitope(s) to which these antibodies bind is unknown. Additionally, the nucleic acid sequence or the amino acid sequence of the variable regions of IgH and IgL chains of these antibodies are not known.

Optionally, isolated antibody-producing cells are then cultured (i.e. grown in media that supports at least one, at least 5 or at least 10 or more cell divisions of the cell) by methods known to one of skill in the art after they have been deposited (see e.g. WO 01/55216).

In some embodiments, the nucleic acids described below may be obtained from a single antibody-producing cell. In certain embodiments, the nucleic acids described below may be obtained more than about 2, more than about 5, more than about 10, more than about 20, more than about 50, more than about 100, more than about 300, more than about 1000, more than about 3000, or more than about 100,000 antibody producing cells.

### Immunoglobulin Heavy Chain-Encoding Nucleic Acids

In some embodiments, nucleic acids encoding the IgH chain variable domain are obtained from the antibody producing cells. These nucleic acids may encode the IgH chain variable domain alone, or may encode a larger fragment of the immunoglobulin heavy chain, such as a heavy chain variable domain and part of the heavy chain constant region, or an entire immunoglobulin heavy chain, optionally including the N-terminal methionine and secretion signal of the immunoglobulin heavy chain.

An immunoglobulin heavy chain-encoding nucleic acid, once obtained from a subject isolated antibody producing cell, is operably linked to an expression polynucleotide that will allow for expression, and optionally secretion of a functional immunoglobulin heavy chain from a host cell. In particular embodiments, therefore, the expression polynucleotide may encode an appropriate region of an immunoglobulin heavy chain, such as a constant domain or a secretion signal peptide to allow a functional immunoglobulin heavy chain to be expressed, and optionally secreted. For example, if the nucleic acid isolated from a cell encodes an immunoglobulin heavy chain variable domain without a constant domain, an appropriate constant domain-encoding polynucleotide, which will optionally encode a secretion signal peptide, will be operably linked to the nucleic acid. In some embodiments, a nucleic acid encoding an entire immunoglobulin heavy chain, including the N-terminal methionine, will be isolated from a cell. In these embodiments, the expression polynucleotide will usually not encode any part of an immunoglobulin heavy chain.

In some embodiments, where the operably linked expression polynucleotide encodes an appropriate region of an immunoglobulin heavy chain, the polynucleotide may encode a region from a different species as compared to the species from which the cell is derived. For example, the appropriate region may be a human, mouse, rabbit or an appropriate region from any mammalian species. If a humanized monoclonal antibody is desired, human sequences may be chosen, whereas if a murinized monoclonal antibody is desired, mouse sequences may be chosen.

In particular embodiments, the immunoglobulin heavy chain-encoding nucleic acid does not encode any part of a viral-derived polypeptide, and encodes a secretion signal peptide sufficient for secretion of the expressed immunoglobulin heavy chain into culture medium. Thus, in exemplary embodiments, the engineered antibody produced by the subject method is not displayed on the surface of a phage. In some cases, the engineered antibody produced by the subject method is not displayed on the surface of the host cell into which the nucleic acids encoding the VL and VH chains are introduced.

### Immunoglobulin Light Chain-Encoding Nucleic Acids

In some embodiments, the nucleic acids encoding an immunoglobulin light chain variable domain is obtained from the antibody-producing cells. A light chain-encoding nucleic acid may encode a light chain variable domain alone, or may encode a larger fragment of an immunoglobulin light chain, such as a light chain variable domain and part of the light chain constant region, or an entire immunoglobulin light chain, optionally including the N-terminal methionine and secretion signal of the immunoglobulin light chain.

An immunoglobulin light chain-encoding nucleic acid, once obtained from the antibody producing cells, is operably linked to an expression polynucleotide that will allow for expression, and optionally secretion of a functional immunoglobulin light chain from a host cell. In some embodiments, therefore, the expression polynucleotide may encode an appropriate region of an immunoglobulin light chain, such as a constant domain or a secretion signal peptide to allow a functional immunoglobulin light chain to be expressed, and optionally secreted. For example, if the nucleic acid isolated from the cell encodes an immunoglobulin light chain variable domain without a constant domain, an appropriate constant domain-encoding polynucleotide, which will optionally encode a secretion signal peptide, will be operably linked to the nucleic acid. In some embodiments, a nucleic acid encoding an entire immunoglobulin light chain, including the N-terminal methionine, will be isolated from a cell. In these embodiments, the expression polynucleotide will usually not encode any part of an immunoglobulin light chain.

In some embodiments, where the operably linked expression polynucleotide encodes an appropriate region of an immunoglobulin light chain, the polynucleotide may encode a region from a different species as compared to the species from which the cell is derived. For example, the appropriate region may be a human, mouse, rabbit or an appropriate region from any mammalian species. If a humanized monoclonal antibody is desired, human sequences may be chosen, whereas if a murinized monoclonal antibody is desired, mouse sequences may be chosen.

In particular embodiments, the immunoglobulin light chain-encoding nucleic acid does not encode any part of a viral-derived polypeptide, and encodes a secretion signal peptide sufficient for secretion of the expressed immunoglobulin heavy chain into culture medium.

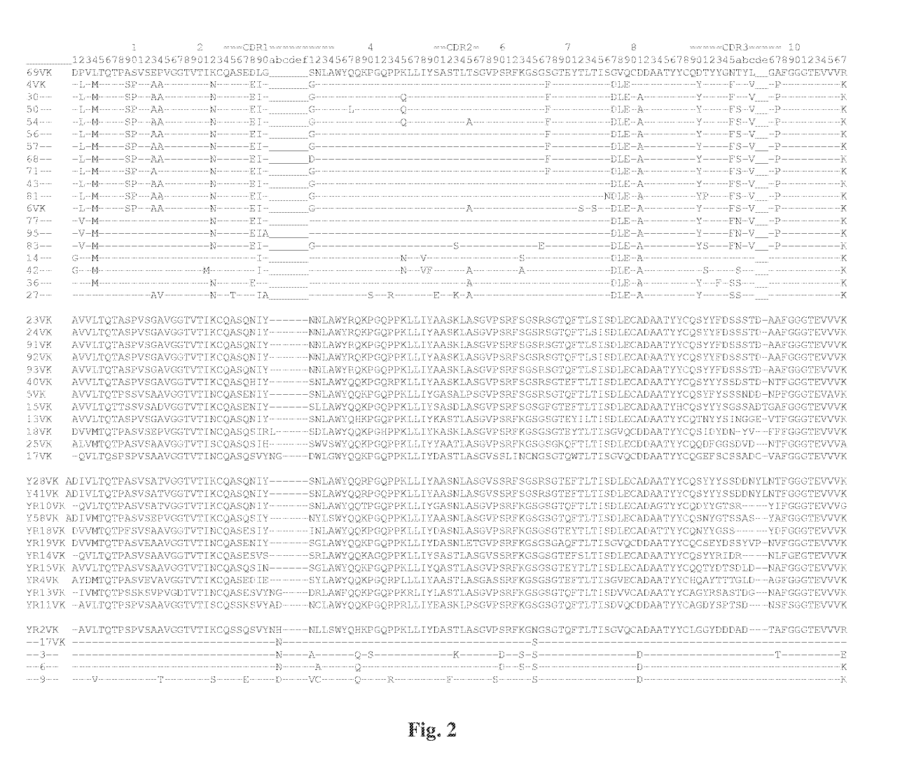

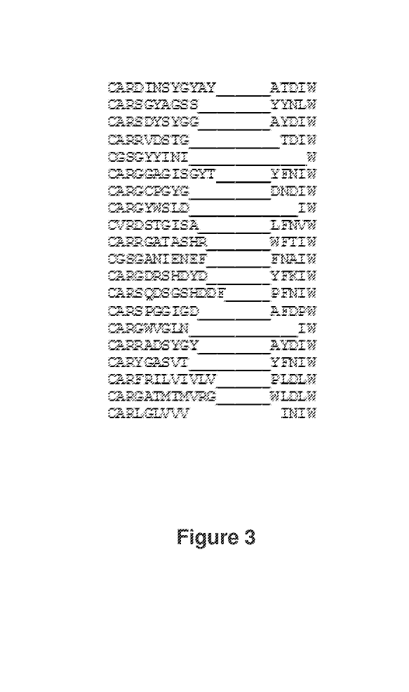

In certain embodiments, the nucleic acids encoding VH and VL chains obtained by the foregoing methods are sequenced and the amino acid sequences are compared to identify a group of related antibodies. This may be done by numbering the amino acid positions of each antibody using a suitable numbering system, such as that provided by Chothia (J Mol Biol 1998; 278: 457-79) or Kabat (1991, Sequences of Proteins of Immunological Interest, DHHS, Washington, D.C.). CDR and/or framework residues may be identified using these methods. The numbered sequences may be aligned by eye, or by employing an alignment program such as one of the CLUSTAL suite of programs (Thompson et al Nucleic Acids Research, 22:4673-4680). The variable regions of antibodies within a related group of antibodies have amino acid sequences that are very similar. For example, the VH or VL domains of antibodies within a related group of antibodies may have amino acid sequences that are at least about 80% identical (e.g., at least 85% identical, at least 90% identical, at least 95% or at least 98% or at least 99% identical), ignoring any gaps or insertions made to facilitate alignment of the sequences. Antibodies within a related group of antibodies have a VL domains that are similar to each other, as well as VH domains that are similar to each other. In other words, in certain embodiments the VH or VL domains of two different related antibodies usually contain up to about ten (i.e., one, two, three, four or five or more) amino acid differences. An amino acid difference may be present at any position of the variable domain, including in any CDR or in any framework region. Certain related antibodies, e.g., related rabbit antibodies have H3 CDRs that are almost identical, as well as L3 CDRs that are almost identical. In these embodiments, any two antibodies that are related will have L3 and H3 CDRs that are each identical in length and have near identical sequences (i.e., that contain 0, 1, 2, 3, 4 or 5 amino acid changes). In other words the L3 CDRs of the two antibodies are identical in length and near identical in sequence and the H3 CDRs of the two antibodies are identical in length and near identical in sequence. Two exemplary sets of related antibodies are shown in FIG. 2, and the sequences of 20 exemplary VH3 regions of unrelated rabbit antibodies are shown for comparison in FIG. 3.

In certain embodiments, the nucleic acid encoding VH chain of an antibody in a group of related antibodies may be paired with the nucleic acid encoding VL chain of another antibody in the group of related antibodies to generate an engineered antibody.

In some embodiments, the sequence of nucleic acids encoding the VH and VL chains of a first antibody of a first antibody producing cell isolated by the methods described above may be identified. The nucleic acids encoding the variable heavy and variable light domains of related antibodies (i.e., antibodies related to the first antibody) may be amplified from the nucleic acids obtained above (i.e., cDNA or amplified VH and VL chain nucleic acid) by using pairs of primers that each contains a primer that is complementary to a CDR-encoding region of the first antibody cDNA. In these embodiments, the method may include: a) obtaining the nucleotide sequences of: i. a heavy chain-encoding nucleic acid that encodes the variable heavy chain of a first antibody of an immunized animal; and ii. a variable light chain-encoding nucleic acid that encodes the light chain of the first antibody; b) obtaining the amino acid sequence of the variable domains of the heavy and light chains of related antibodies, using: i. a first primer pair that includes a first primer that is complementary to a CDR-encoding region of the heavy chain-encoding nucleic acid; and ii. a second primer pair that includes a second primer that is complementary to a CDR-encoding region of the light chain-encoding nucleic acid.

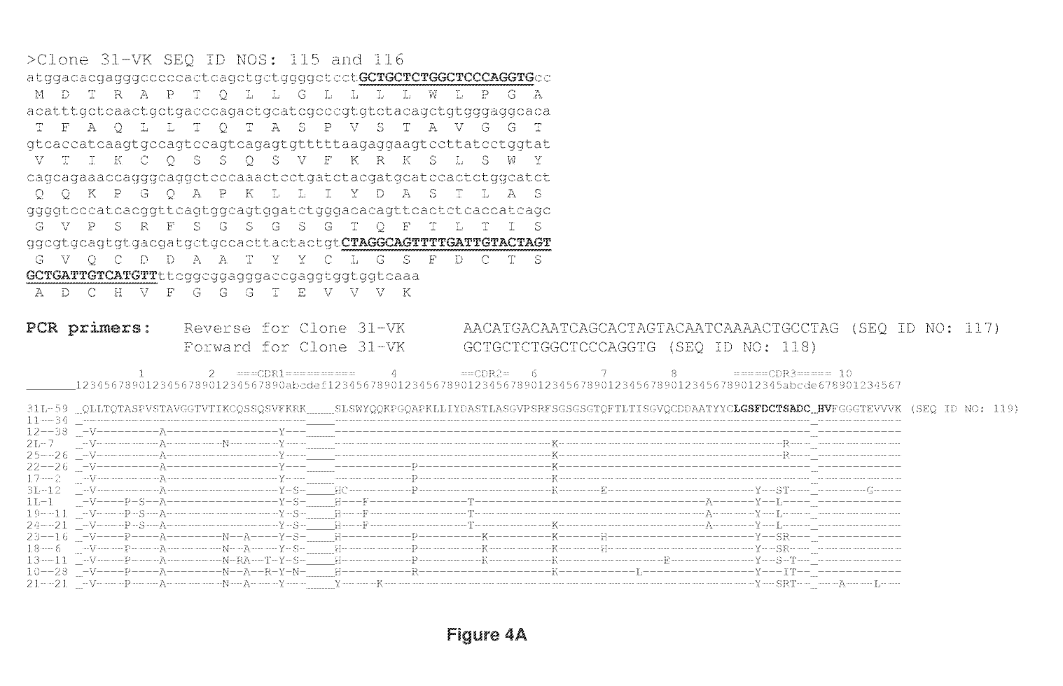

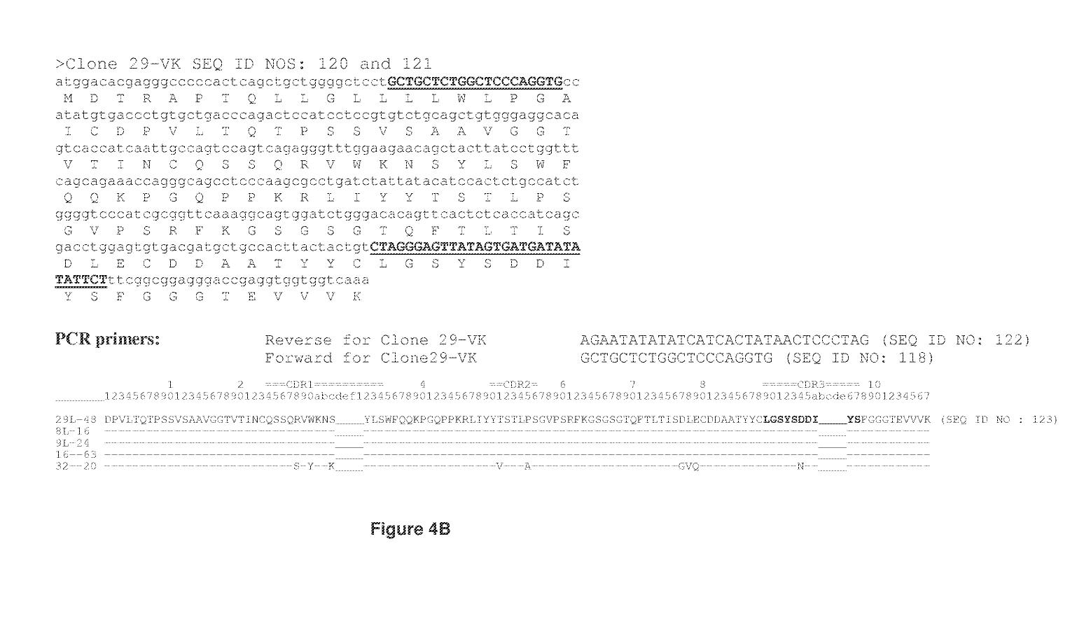

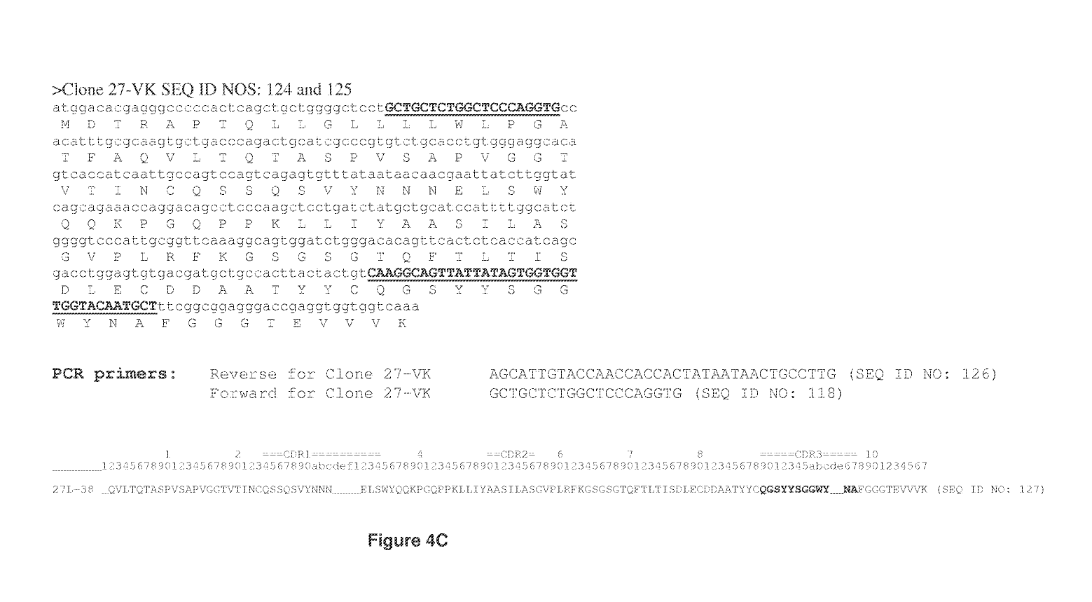

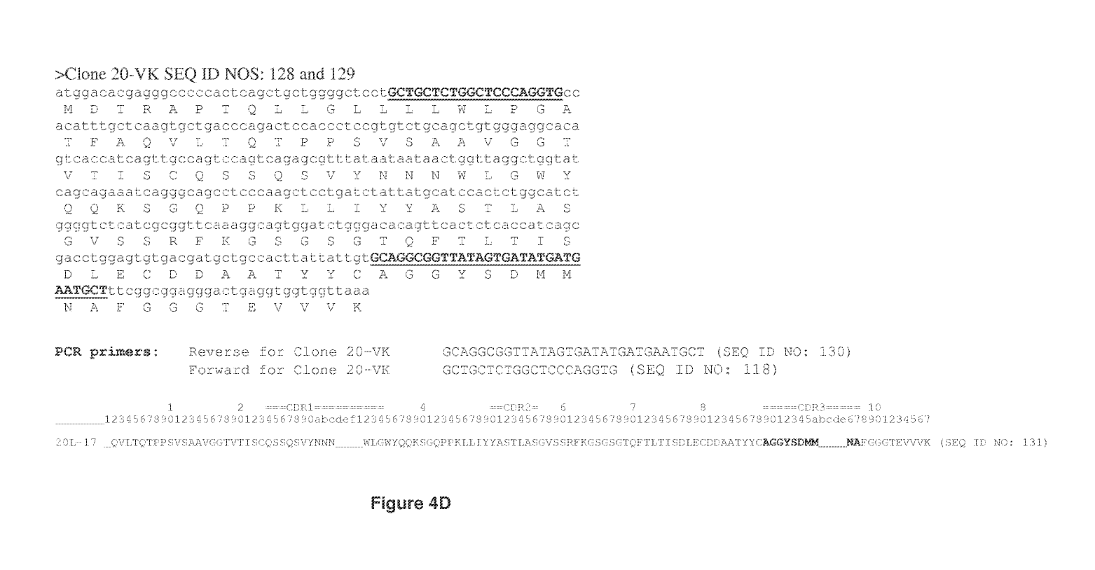

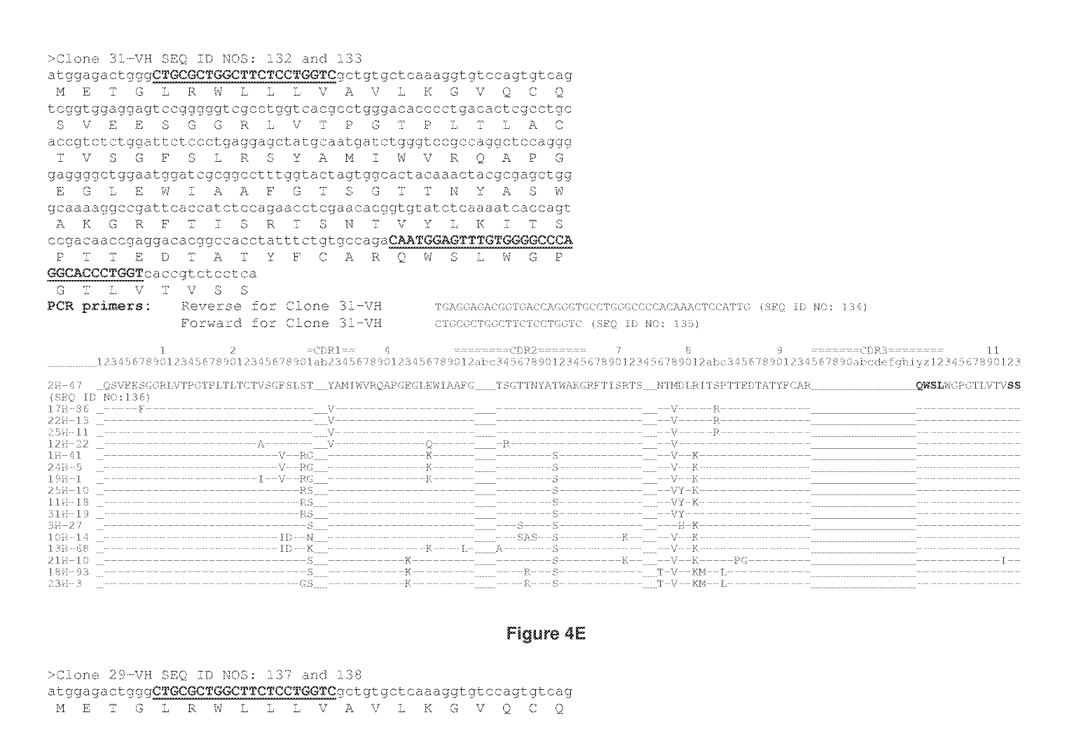

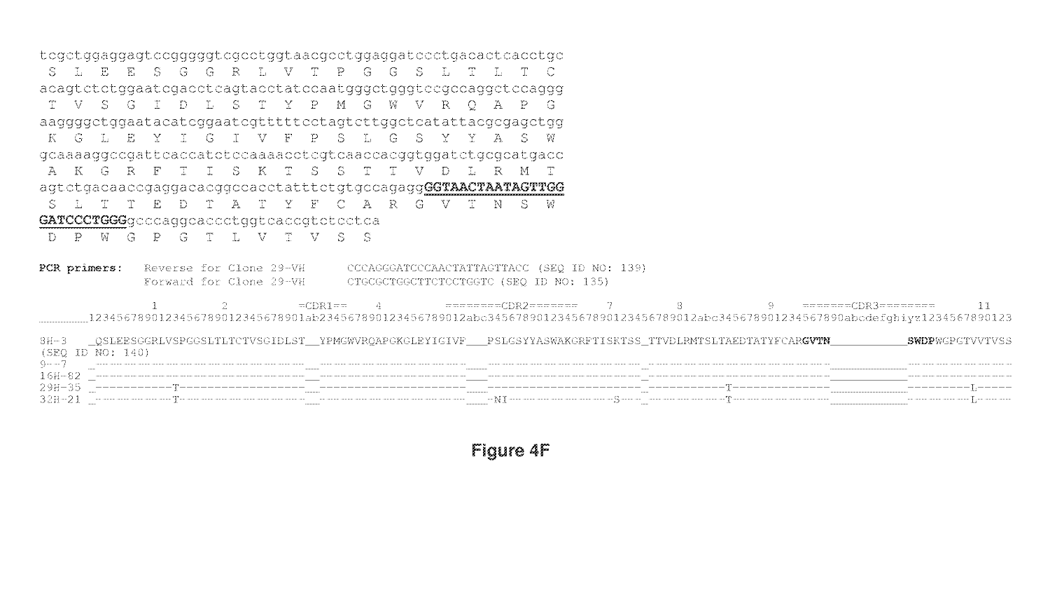

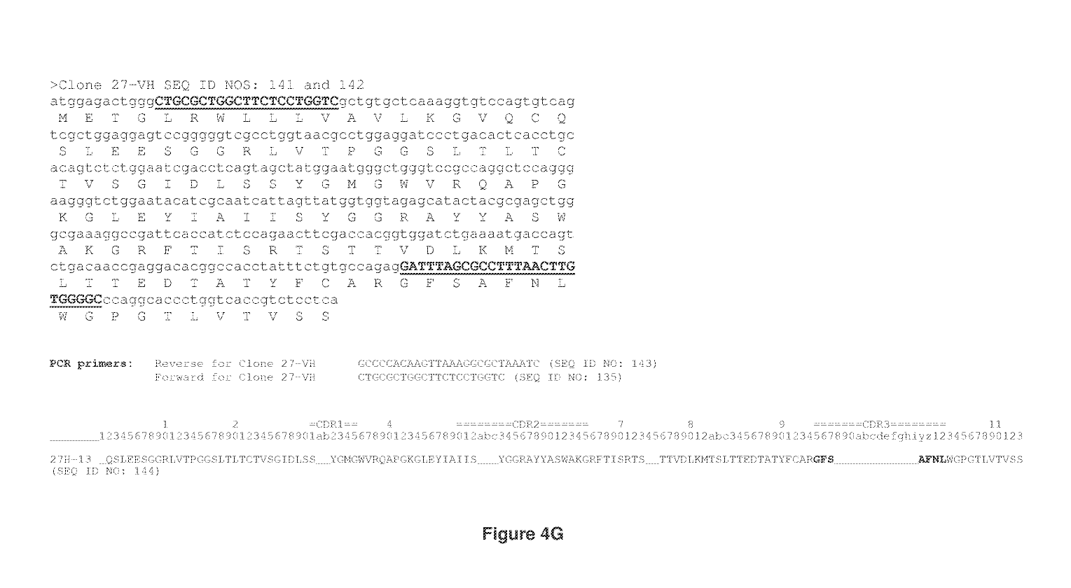

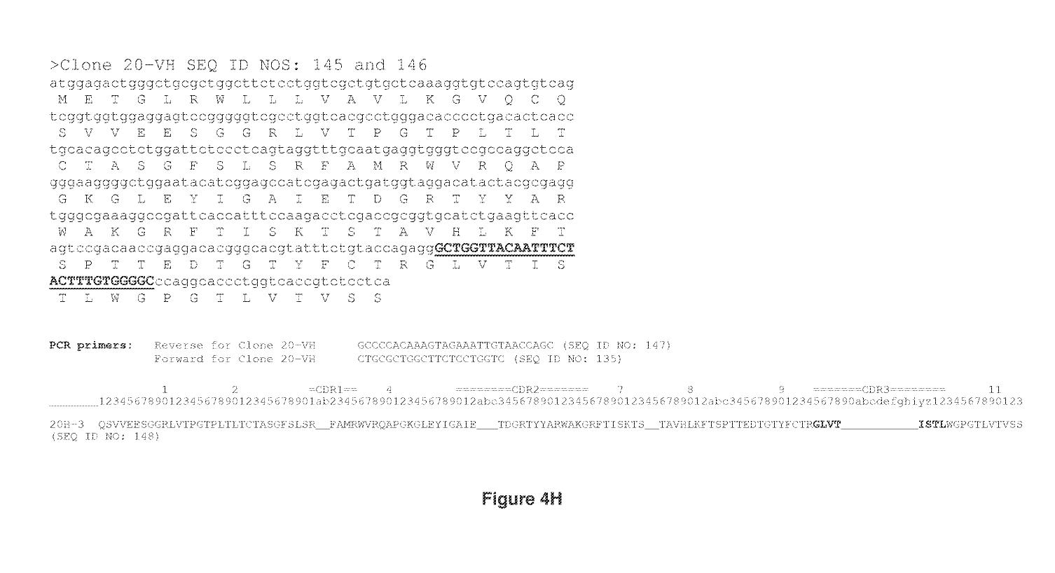

The heavy chain CDR-specific primer may be complementary to the sequence that encodes the CDR1, CDR2 or CDR3 region of the heavy chain of the first antibody and, likewise, the light chain CDR-specific primer may be complementary to the sequence that encodes the CDR1, CDR2 or CDR3 region of the light chain of the first antibody. In certain embodiments, a particular CDR-specific primer may be chosen because the CDR sequence to which it binds may be known to be less variable than other CDR sequences. For example, in the examples shown in FIG. 4A-H, the light chain was amplified using primers complementary to the CDR3-encoding region, and the heavy chain were amplified using primers complementary to the CDR3-encoding region.

In these embodiments, an amplification reaction may be carried out using cDNA made from the antibody producing cells isolated by methods described above as a template. The amplification reaction may be carried out using nucleic acid obtained from single cells (or cultures of the same) or nucleic acid obtained from pooled cells (e.g., pools of different antibody-producing cells). Pools may contain cDNA from at least 10, at least 50, at least 100, or at least 500, at least 1000, or more different antibody cells that bind to the same antigen, for example.

As noted above, in certain embodiments, nucleic acid encoding the heavy and light chains of related antibodies may be obtained by PCR amplification using CDR-specific primers. In these embodiments, the heavy and light chains may be combined with each other, e.g., systematically or at random, to provide antibodies that are not produced by the immunized animal, i.e., to provide a library of antibodies that contains antibodies that are neither the first antibody or an antibody related to the first antibody by lineage. Since the first antibody and related antibodies are related by lineage and contain minimal sequence differences relative to one another, the resultant antibodies in the library—which contain new combinations of heavy and light chains relative to the first and related antibodies—would be functional (i.e., would be expected to bind to the same antigen as the first antibody). The antibody library can be screened using standard methods, some of which are described below, to identify an antibody with a desired activity. This antibody may contain a heavy chain from a first antibody and a light chain from a second antibody, where the first and second antibodies are different antibodies that are related by lineage.

As would be readily apparent, the pairing of the heavy and light chains may be done many different ways, e.g., systematically or randomly and, in certain cases, may be done using pooled nucleic acid. In particular embodiments, the pairing may involve systematically combining the variable domains of the heavy and light chains of the first antibody and the further antibodies to produce a library of antibodies that contains at least 50% of all possible combinations of variable domains. In other embodiments, the pairing step may involve: i. introducing: a) a pool of heavy chain-encoding nucleic acid that encodes a plurality of different amplified heavy chain variable domains and b) a pool of light chain-encoding nucleic acid that encodes a plurality of different amplified light chain variable domains, into population of cells, and ii. selecting cells that contain both a heavy chain-encoding nucleic acid and a light chain-encoding nucleic acid, to produce a library of cells that produce the library of antibodies. As would be apparent, a number of different cloning strategies may be employed to produce the pools of nucleic acids.

The above-described CDR-anchored method for identifying related antibodies has been described in detail in U.S. Application Ser. No. 61/151,052, filed Feb. 9, 2009. The methods described in U.S. Application Ser. No. 61/151,052 are incorporated herein by reference.

### Expression Cassette Pairs

Expression cassettes pairs, when introduced into host cells, provide for expression of the immunoglobulin heavy and light chains in the host cells. In particular embodiments, each expression cassette of a pair is more than about 0.5 kb in length, more than about 1.0 kb in length, more than about 1.5 kb in length, more than about 2 kb in length, more than about 4 kb in length, more than about 5 kb in length, and is usually less than 10 kb in length. An expression cassette may be linear, or encompassed in a circular vector.

Each of the heavy and light chain expression polynucleotides described above will typically further include expression control DNA sequences operably linked to the immunoglobulin coding sequences to form heavy and light chain expression cassettes. In some embodiments, the expression control sequences will be eukaryotic promoter capable of directing expression of the immunoglobulin heavy or light chain polypeptide in eukaryotic host cells. In certain embodiments, a human cytomegalovirus (HCMV) promoter and/or enhancer and/or terminator is used to direct expression of the polypeptides in mammalian cells. Suitable promoters, terminators, and translational enhancers suitable for expression of immunoglobulin heavy and light chains are known in the art, and many are discussed in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). Suitable promoters include SV40 elements, as described in Dijkema et al., EMBO J. (1985) 4:761; transcription regulatory elements derived from the LTR of the Rous sarcoma virus, as described in Gorman et al., Proc. Nat'l Acad. Sci USA (1982) 79:6777; transcription regulatory elements derived from the LTR of human cytomegalovirus (CMV), as described in Boshart et al., Cell (1985) 41:521; hsp70 promoters, (Levy-Holtzman, R. and I. Schechter (Biochim. Biophys. Acta (1995) 1263: 96-98) Presnail, J. K. and M. A. Hoy, (Exp. Appl. Acarol. (1994) 18: 301-308)) and the like.

In some embodiments, the heavy and light chain expression cassettes are linear expression cassettes, or are present on a circular nucleic acid (e.g. a circular vector, for example a plasmid). Linear expression cassettes are typically not inserted into a circular vector and are not otherwise associated with vector sequences such as an origin of replication, or vector backbone. In certain embodiments, however, the linear expression cassette may also provide for expression of a selectable marker. Suitable vectors and selectable markers are well known in the art and discussed in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.). A variety of different genes have been employed as selectable markers, and the particular gene employed in the subject vectors as a selectable marker is chosen primarily as a matter of convenience. Known selectable marker genes include: the thymidine kinase gene, the dihydrofolate reductase gene, the xanthine-guanine phosphoribosyl transferase gene, CAD, the adenosine deaminase gene, the asparagine synthase gene, the antibiotic resistance genes, e.g. tet^{r}, amp^{r}, Cm^{r} or cat, kan^{r} or neo^{r} (aminoglycoside phosphotransferase genes), the hygromycin B phosphotransferase gene, and the like.

In particular embodiments, the linear expression cassette is a non-integrative polynucleotide, i.e., it does not integrate into a genome of a host cell, and, as such, typically does not contain recombination sites or flanking sequences to facilitate homologous recombination.

In certain embodiments, the heavy and light chain coding sequences are present on the same nucleic molecule, and expression of the two chains may be accomplished by using a single promoter and an internal ribosome entry site (IRES) between the two coding sequences. Such constructs are known to those of skill in the art, see, e.g., Dirks, 1993 (Gene 128: 247-9).

In some embodiments, an antibody producing cell, usually a single cell, is deposited into a well of a plate in a minimal volume (in about 0.1 μl, about 0.5 μl, about 1 μl, or about 5 μl), and polynucleotides encoding a immunoglobulin heavy chain variable domain and an immunoglobulin light chain variable domain are obtained, e.g. harvested, isolated, amplified, etc., from the cell. In certain cases, a population of a plurality of antibody producing cells isolated by the above described procedures is used for obtaining a plurality of first nucleic acids encoding the IgH variable region and a plurality of second nucleic acids encoding the IgL variable region. In particular embodiments this is done using an amplification procedure, such as the polymerase chain reaction. For example, once antibody-producing cells have been isolated, RNA is recovered from the cells by established methods, such as the method of Rappolee et al. (J. Cell Biochem. 39:1-11, 1989), or a scaled-down version of the method of Gonda et al. (J. Virol. 61: 2754-2763, 1987) and once RNA has been recovered, cDNA is made. Many methods for constructing cDNA from RNA are well known in the art, such as those described by Sambrook et al. (Sambrook, Fritsch and Maniatis, Molecular Cloning—A Laboratory Manual, Cold Spring Harbor Laboratory Press, 1989).

Sequences encoding heavy and light chains may be amplified from the cDNA using techniques well known in the art, such as Polymerase Chain Reaction (PCR). See Mullis, U.S. Pat. No. 4,683,195; Mullis et al., U.S. Pat. No. 4,683,195; Polymerase Chain Reaction: Current Communication in Molecular Biology, Cold Springs Harbor Press, Cold Spring Harbor, N.Y., 1989. Briefly, cDNA segments encoding the variable domain of the antibody are exponentially amplified by performing sequential reactions with a DNA polymerase. The reaction is primed by a 5′ and a 3′ DNA primer. In some embodiments, the 3′ antisense primer corresponding to a DNA sequence in the constant (or joining) region of the immunoglobulin chain and the 5′ primer (or panel of related primers) corresponding to a DNA sequence in the variable region of the immunoglobulin chain. This combination of oligonucleotide primers has been used in the PCR amplification of murine immunoglobulin cDNAs of unknown sequence (see Sastry et at., Proc Natl. Acad. Sci. 86:5728-5732, 1989 and Orlandi et al., Proc. Natl. Acad. Sci. 86:3833-3837, 1989).

Alternatively, an “anchored polymerase chain reaction” may be performed (see Loh et al., Science 243:217-220, 1989). In this procedure, the first strand cDNA is primed with a 3′ DNA primer as above, and a poly (dG tail) is then added to the 3′ end of the strand with terminal deoxynucleotidyl transferase. The product is then amplified by PCR using the specific 3′ DNA primer and another oligonucleotide consisting of a poly(dC) tail attached to a sequence with convenient restriction sites. In some embodiments, however, the entire polynucleotide encoding a heavy or light chain is amplified using primers spanning the start codons and stop codons of both of the immunoglobulin cDNAs, however, depending on the amplification products desired, suitable primers may be used. Exemplary primers for use with rabbit antibody-producing cells are as follows: heavy chain, 5′ end (CACCATGGAGACTGGGCTGCGCTGGCTTCTCCTGGTCGCTGTG; SEQ ID NO: 149); heavy chain, 3′ end (CTCCCGCTCTCCGGGTAAATGAGCGCTGTGCCGGCGA; SEQ ID NO: 150); light chain kappa, 5′ end (CAGGCAGGACCCAGCATGGACACGAGGGCCCCCACT; SEQ ID NO: 151); and L kappa, 3′ end (TCAATAGGGGTGACTGTTAGAGCGAGACGCCTGC; SEQ ID NO: 152). Suitable restriction sites and other tails may be engineered into the amplification oligonucleotides to facilitate cloning and further processing of the amplification products. Amplification procedures using nested primers may also be used, where such nested primers are well known to one of skill in the art.

Once polynucleotides encoding immunoglobulin heavy and light chain variable domains are amplified from a cell, they are assembled with appropriate antibody domains and/or regulatory sequences to form an expression cassette.

In general, the nucleic acids encoding IgH and IgL chains that form an expression cassette pair are not mutagenized. In other words, the nucleic acids encoding IgH and IgL chains are identical or substantially identical to those present in the antibody-producing cells. In certain cases, the subject sequence of nucleic acids encoding IgH and IgL chains may have one or two nucleotide base difference from the sequence of those present in the antibody-producing cells due to the amplification process by which the subject nucleic acids were obtained.

In order to assemble an expression cassette, i.e., to operably link the coding sequences with any other coding or regulatory sequences, standard recombinant DNA technology (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.) may be used. Several methods are known in the art for producing antibody-encoding nucleic acids, including those found in U.S. Pat. Nos. 6,180,370, 5,693,762, 4,816,397, 5,693,761 and 5,530,101. One PCR method utilizes “overlapping extension PCR” (Hayashi et al., Biotechniques. 1994: 312, 314-5) to create expression cassettes for the heavy and light chain encoding nucleic acids. In this method multiple overlapping PCR reactions using the cDNA product obtained from the antibody producing cell and other appropriate nucleic acids as templates generates an expression cassette.

Depending on the constant regions and other regions used, several types of antibody that are known in the art may be made by this method. As well as full length antibodies, antigen-binding fragments of antibodies may be made. These fragments include, but are not limited to, Fab, Fab′ and F(ab′)2, Fd, single-chain Fvs (scFv), single-chain immunoglobulins (e.g., wherein a heavy chain, or portion thereof, and light chain, or portion thereof, are fused), disulfide-linked Fvs (sdFv), diabodies, triabodies, tetrabodies, scFv minibodies, Fab minibodies, and dimeric scFv and any other fragments comprising a VL and a VH domain in a conformation such that a specific antigen binding region is formed. Antibody fragments, including single-chain antibodies, may comprise the variable region(s) alone or in combination with the entire or partial of the following: a heavy chain constant domain, or portion thereof, e.g., a CH1, CH2, CH3, transmembrane, and/or cytoplasmic domain, on the heavy chain, and a light chain constant domain, e.g., a Ckappa or Clambda domain, or portion thereof on the light chain. Also included are any combinations of variable region(s) and CH1, CH2, CH3, Ckappa, Clambda, transmembrane and cytoplasmic domains.

Production of circular vectors for expression of antibodies is well known in the art (Ausubel, et al, Short Protocols in Molecular Biology, 3rd ed., Wiley & Sons, 1995; Sambrook, et al., Molecular Cloning: A Laboratory Manual, Second Edition, (1989) Cold Spring Harbor, N.Y.). Certain recombination-based methods, e.g. GATEWAY™ (InVitrogen, Carlsbad, Calif.)), CREATOR™ (Clontech, Palo Alto, Calif.) or ET cloning (Muyrers et al, Nucleic Acids Res. 27:1555-7 (1999)) methodologies may also be used in the production of expression cassettes.

In certain embodiments, the plurality of first nucleic acids encoding the IgH variable region may be cloned into a vector with a selectable marker, and the plurality of second nucleic acids encoding the IgL variable region may be cloned into another vector with a different selectable marker. Thus, in such embodiments, the expression cassette pair is present in a separate first and a second polynucleotide.

In alternative examples, a first nucleic acid encoding an IgH variable chain of the plurality of first nucleic acids and a second nucleic acid encoding an IgL variable chain of the plurality of second nucleic acids may be cloned into a single vector. In this embodiment, the first and the second nucleic acids of the expression cassette pair are present in a single polynucleotide. In embodiments where the first nucleic acid encoding an IgH variable chain of the plurality of first nucleic acids and a second nucleic acid encoding an IgL variable chain of the plurality of second nucleic acids are cloned into a single vector, the first and second nucleic acids may be randomly paired or systematically paired.

For random pairing, the first and second nucleic acids may be amplified using primers with appropriate restriction sites for cloning into a single vector. The expression cassette pairs may be obtained by a single ligation reaction using three nucleic acid components, i.e., vector, IgH chain encoding nucleic acid and IgL chain encoding nucleic acid. In other embodiments, the first and second nucleic acids may be sequentially cloned into a vector.

For systematic pairing, the first and second nucleic acids may first be cloned into two separate vectors to generate IgH clones and IgL clones, respectively. For example, a first nucleic acid encoding IgH chain may be obtained from IgH clone 1 and a second nucleic acid encoding IgL chain may be obtained from IgL clone 2 and then cloned into a single vector. Systematic pairing is best understood using an example in which the isolated antibody producing cells are deposited as single cells in the wells of a microtiter plate. In this example, the nucleic acids encoding the IgH and the IgL chain in each cell may be obtained using appropriately designed primers. Subsequently, nucleic acid encoding IgH obtained from well 1 is cloned into a vector and nucleic acid encoding IgL obtained from well 2 is cloned into the same vector. Similarly, nucleic acid encoding IgH obtained from well 2 is cloned into a vector and nucleic acid encoding IgL obtained from well 3 is cloned into the same vector, and so on.

### Expression of Immunoglobulin Heavy and Light Chains

In particular embodiments, immunoglobulin heavy and light chain expression cassette pairs are introduced directly into a host cell, and the cell incubated under conditions sufficient to induce expression of the encoded immunoglobulin heavy and light chains.

In particular examples, the expression cassette pairs may be produced randomly. For example, in a scenario where a plurality of first nucleic acids encoding the IgH variable regions are, e.g., cloned into a plurality of vector A and a plurality of second nucleic acids encoding the IgL variable regions are, e.g., cloned into a plurality of vector B, the pluralities of vectors A and B are mixed together in equimolar amounts to produce expression cassette pairs.

In other cases, the first and the second nucleic acids of the expression cassette pair may be systematically paired. Systematic pairing is described in the following example: Ten nucleic acids encoding the IgH may be, e.g., cloned into vector A and each of the clones are separated into individual IgH clones 1-10. Similarly, ten nucleic acids encoding the IgL may be, e.g., cloned into vector B and each of the clones are separated into individual IgL clones 1-10. In this example, a first expression cassette pair may include IgH clone 1 and IgL clone 2; a second expression cassette pair may include IgH clone 2 and IgL clone 3; a third expression cassette pair may include IgH clone 3 and IgL clone 4, and so on.

In embodiments where the expression cassette pairs are present on separate first and second polynucleotides, the polynucleotides have different selectable markers to ensure the selection of cells that contain both members of the pair.

Any cell suitable for expression of expression cassettes may be used as a host cell. Usually, a mammalian host cell line that does not ordinarily produce antibodies is used, examples of which are as follows: monkey kidney cells (COS cells), monkey kidney CV1 cells transformed by SV40 (COS-7, ATCC CRL 165 1); human embryonic kidney cells (HEK-293, Graham et al. J. Gen Virol. 36:59 (1977)); baby hamster kidney cells (BHK, ATCC CCL 10); chinese hamster ovary-cells (CHO, Urlaub and Chasin, Proc. Natl. Acad. Sci. (USA) 77:4216, (1980); mouse sertoli cells (TM4, Mather, Biol. Reprod. 23:243-251 (1980)); monkey kidney cells (CV1 ATCC CCL 70); african green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (hep G2, HB 8065); mouse mammary tumor (MMT 060562, ATCC CCL 51); TR1 cells (Mather et al., Annals N.Y. Acad. Sci 383:44-68 (1982)); NIH/3T3 cells (ATCC CRL-1658); and mouse L cells (ATCC CCL-1). Additional cell lines will become apparent to those of ordinary skill in the art. A wide variety of cell lines are available from the American Type Culture Collection, 10801 University Boulevard, Manassas, Va. 20110-2209.

Methods of introducing linear nucleic acids into cells are well known in the art. Suitable methods include electroporation, particle gun technology, calcium phosphate precipitation, direct microinjection, and the like. The choice of method is generally dependent on the type of cell being transformed and the circumstances under which the transformation is taking place (i.e. in vitro, ex vivo, or in vivo). A general discussion of these methods can be found in Ausubel, et al, Short Protocols in Molecular Biology, 3rd ed., Wiley & Sons, 1995. In some embodiments, lipofectamine and calcium mediated gene transfer technologies are used. Methods for introducing circular nucleic acids are also well known in the art and discussed in Ausubel, above.

After introduction of expression cassette pairs into cells, the cells are typically incubated, normally at 37° C., sometimes under selection, for a period of about 1-24 hours in order to allow for the expression of the antibody. In particular embodiments, the antibody is typically secreted into the supernatant of the media in which the cells are growing.

In certain embodiments, the library of engineered-antibody producing cells may have a complexity of at least 2, at least 5, at least 10, at least 20, at least 50, at least 80, at least 100, at least 300, at least 500, at least 1000, at least 10,000, at least 100,000, or at least 1000,000, or more, wherein each unique cell produces a unique engineered antibody.

### Methods of Screening a Library of Engineered-Antibody Producing Cells

A method of screening the library of engineered-antibody producing cells is provided. In general, this method involves producing a library of engineered-antibody producing cells using the method described above and screening the library for cells that produce engineered-antibodies using one or a combination of a variety of assays. In general, these assays are functional assays, and may be grouped as follows: assays that detect an antibody's binding affinity or specificity, and assays that detect the ability of an antibody to inhibit a process.

### Binding Assays

In these assays, antibodies produced by the library of engineered-antibody producing cells are tested for their ability to bind specifically to an antigen. The term “specifically” in the context of antibody binding, refers to high avidity and/or high affinity binding of an antibody to a specific antigen. In some embodiments, the specific antigen is an antigen (or a fragment or subfraction of an antigen) used to immunize the animal host from which the antibody-producing cells were isolated. Specific binding of an antibody to an antigen or fragment thereof is stronger than binding of the same antibody to other antigens which were not used to immunize the animal. Antibodies which bind specifically to a polypeptide antigen may be capable of binding other polypeptides at a weak, yet detectable, level (e.g., 10% or less of the binding shown to the polypeptide of interest). Such weak binding or background binding is readily discernible from the specific antibody binding to a subject polypeptide, e.g. by use of appropriate controls. In general, specific antibodies bind to an antigen with a binding affinity of 10⁻⁷ M or more, e.g., 10⁻⁸ M or more (e.g., 10⁻⁹ M, 10⁻¹⁰, 10⁻¹¹, etc.). In general, an antibody with a binding affinity of 10⁻⁶ M or less is not useful in that it will not bind an antigen at a detectable level using conventional methodology currently used.

Typically, in performing a screening assay, antibody samples produced by a library of engineered-antibody producing cells are deposited onto a solid support in a way that each antibody can be identified, e.g. with a plate number and position on the plate, or another identifier that will allow the identification of the host cell culture that produced the antibody.

Subject antibodies may be screened for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, and protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally involve lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads).

Western blot analysis generally involves preparation of protein samples followed by electrophoresis of the protein samples in a polyacrylamide gel (e.g., 8%-20% SDS-PAGE depending on the molecular weight of the antigen), and transfer of the separated protein samples from the polyacrylamide gel to a membrane such as nitrocellulose, PVDF or nylon. Following transfer, the membrane is blocked in blocking solution (e.g., PBS with 3% BSA or non-fat milk), washed in washing buffer (e.g., PBS-Tween 20), and incubated with primary antibody (the antibody of interest) diluted in blocking buffer. After this incubation, the membrane is washed in washing buffer, incubated with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I), and after a further wash, the presence of the antigen may be detected. One of skill in the art would be knowledgeable as to the parameters that can be modified to increase the signal detected and to reduce the background noise.

ELISAs involve preparing antigen, coating the well of a 96-well microtiter plate with the antigen, adding the antibody to be assayed conjugated to a detectable compound such as an enzymatic substrate (e.g., horseradish peroxidase or alkaline phosphatase) to the well and incubating for a period of time, and detecting the presence of the antigen. In ELISAs the antibody to be assayed does not have to be conjugated to a detectable compound; instead, a second antibody (which recognizes the antibody to be assayed) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art.

The binding affinity of an antibody to an antigen and the off-rate of an antibody-antigen interaction can be determined by competitive binding assays. One example of a competitive binding assay is a radioimmunoas say comprising the incubation of labeled antigen (e.g., 3H or 125I) with the antibody of interest in the presence of increasing amounts of unlabeled antigen, and the detection of the antibody bound to the labeled antigen. The affinity of the antibody of interest for a particular antigen and the binding off-rates can be determined from the data by Scatchard plot analysis. Competition with a second antibody can also be determined using radioimmunoassays. In this case, the antigen is incubated with antibody to be assayed conjugated to a labeled compound (e.g., 3H or 125I) in the presence of increasing amounts of an unlabeled second antibody.

Subject antibodies may be screened using immunocytochemistry methods on cells (e.g., mammalian cells, such as CHO cells) transfected with a vector enabling the expression of an antigen or with vector alone using techniques commonly known in the art. Antibodies that bind antigen transfected cells, but not vector-only transfected cells, are antigen specific.

In certain embodiments, however, the assay is an antigen capture assay, and an array or microarray of antibodies may be employed for this purpose. Methods for making and using microarrays of polypeptides are known in the art (see e.g. U.S. Pat. Nos. 6,372,483, 6,352,842, 6,346,416 and 6,242,266).

### Inhibitor Assays

In certain embodiments, the assay measures the specific inhibition of an interaction between a first compound and a second compound by the subject antibody (e.g. two biopolymeric compounds) or the specific inhibition of a reaction by the subject antibody (e.g. an enzymatic reaction). In an interaction inhibition assay, one interaction substrate, usually a biopolymeric compound such as a protein e.g. a receptor, may be bound to a solid support in a reaction vessel. Antibody is added to the reaction vessel followed by a detectable binding partner for the substrate, usually a biopolymeric compound such as a protein e.g. a radiolabeled ligand for the receptor. After washing the vessel, interaction inhibition may be measured by determining the amount of detectable binding partner present in the vessel. Interaction inhibition occurs when binding of the binding partner is reduced greater than about 20%, greater than about 50%, greater than about 70%, greater than about 80%, or greater than about 90% or 95% or more, as compared to a control assay that does not contain the subject antibody. Although some of the procedures described herein specify the use of a single cell or antibody, it is understood that these procedures also cover the use of a plurality of antibodies or cells.

In a reaction inhibition assay, an enzyme may be bound to a solid support in a reaction vessel. Subject antibody is usually added to the reaction vessel followed by a substrate for the enzyme. In certain embodiments, the products of the reaction between the enzyme and the substrate are detectable, and, after a certain time, the reaction is usually stopped. After the reaction has been stopped, reaction inhibition may be measured by determining the level of detectable reaction product present in the vessel. Reaction inhibition occurs when the rate of the reaction is reduced greater than about 20%, greater than about 50%, greater than about 70%, greater than about 80%, or greater than about 90% or 95% or more, as compared to a control assay that does not contain antibody.

In Vivo Assays

In certain embodiments the antibodies produced by the library of engineered-antibody producing cells are tested in vivo. In general, the method involves administering a subject antibody to an animal model for a disease or condition and determining the effect of the antibody on the disease or condition of the model animal. In vivo assays generally include controls, where suitable controls include a sample in the absence of the antibody. Generally a plurality of assay mixtures is run in parallel with different antibody concentrations to obtain a differential response to the various concentrations. Typically, one of these concentrations serves as a negative control, i.e. at zero concentration or below the level of detection.

A subject antibody of interest is one that modulates, i.e., reduces or increases a symptom of the animal model disease or condition by at least about 10%, at least about 20%, at least about 25%, at least about 30%, at least about 35%, at least about 40%, at least about 45%, at least about 50%, at least about 55%, at least about 60%, at least about 65%, at least about 70%, at least about 80%, at least about 90%, or more, when compared to a control in the absence of the antibody. In general, a subject antibody of interest will cause a subject animal to be more similar to an equivalent animal that is not suffering from the disease or condition. Antibodies that have therapeutic value that have been identified using the methods and compositions of the invention are termed “therapeutic” antibodies.

### Compositions

A composition of a library of engineered-antibody producing cells, produced by the method described above, is provided. In general, the subject library includes cells that produce antibodies comprising non-naturally paired IgH and IgL variable chains. The subject cells include expression cassette pairs, each expression cassette pair includes a first nucleic acid encoding an immunoglobulin heavy chain variable region and a second nucleic acid encoding an immunoglobulin light chain variable region. As noted above, the first and the second nucleic acids are obtained from a plurality of antibody producing cells from a first animal immunized with an antigen; the first and second nucleic acids are not paired together in a cell of the plurality of antibody producing cells, and the plurality of antibody producing cells express antibodies that bind to the antigen.

In some embodiments, the subject library comprises at least fifty cells. In other embodiments, the subject library comprises at least fifty cells, at least hundred cells, at least thousand cells or more.

In some embodiments, the subject library includes cells that produce antibodies that bind to the antigen. In some cases, the subject library includes at least 2, at least 5, at least 10, at least 30, at least 100, at least 300, at least 1000, or more cells that produce antibodies that bind to the antigen.

In another embodiment, a composition of a plurality of engineered-antibodies is provided. In other embodiments, a composition of an engineered antibody is provided.

These composition are usually contained in microtiter plates, which may be of a 24 well, 48 well, 96 well, 386 well or 1544 well format, and each plate is usually labeled with a unique identifier such that each sample will have a unique name, e.g. based on the name of the plate and the coordinates of the sample within the plate.

### Methods of Identifying Nucleic Acids Encoding an Engineered-Antibody of Interest

A method of identifying nucleic acid encoding a engineered-antibody of interest is provided. In general, the method involves: (a) immunizing an animal with an antigen; (b) producing a library of engineered-antibody producing cells antibodies as described above; (c) screening the library to identify cells producing an engineered-antibody of interest; and (d) identifying nucleic acids encoding the engineered-antibody of interest.

Since the host cell expressing the antibody of interest contains the immunoglobulin heavy and light chain-encoding expression cassettes, the nucleic acids encoding the monoclonal antibody of interest may be identified if the host cell expressing the antibody of interest is identified. As such, the subject nucleic acids may be identified by a variety of methods known to one of skill in the art. Similar methods are used to identify host cell cultures in antibody production using hybridoma technology (Harlow et al., Antibodies: A Laboratory Manual, First Edition (1988) Cold spring Harbor, N.Y.).

For example, upon identifying an antibody of interest, the host cell expressing the antibody of interest may be identified using a “look-up” table which lists, for every antibody sample, the corresponding host cell culture. In certain other embodiments, a look-up table containing antibody library sample identifiers, corresponding expression cassette library sample identifiers and/or host cell identifiers may be used to identify the subject nucleic acids.

Once identified, the nucleic acids encoding an antibody of interest may be recovered, characterized and manipulated using techniques familiar to one of skill in the art (Ausubel, et al, Short Protocols in Molecular Biology, 3rd ed., Wiley & Sons, (1995) and Sambrook, et al, Molecular Cloning: A Laboratory Manual, Third Edition, (2001) Cold Spring Harbor, N.Y.).

### Methods of Producing an Engineered-Antibody of Interest

Several methods of producing an engineered-antibody of interest are provided. In general, these methods involve incubating a host cell containing a nucleic acid encoding a engineered-antibody of interest under conditions sufficient for production of the antibody.

In some embodiments, the methods of producing a monoclonal antibody of interest involve transferring identified expression cassettes for an engineered-antibody of interest into a suitable expression vector, and transferring the vector into a host cell to provide for expression of the engineered-antibody.

In some embodiments, the subject methods involve transferring at least the variable domain-encoding sequences from the identified heavy and light chains into vectors suitable for their expression in immunoglobulin heavy and light chains. Suitable constant domain-encoding sequences and/or other antibody domain-encoding sequences may be added to the variable domain-encoding sequences at this point. These nucleic acid modifications may also allow for humanization of the subject antibody.

A variety of host-expression vector systems may be utilized to express a subject engineered-antibody. These include but are not limited to microorganisms such as bacteria (e.g., E. coli, B. subtilis) transformed with recombinant DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells etc.) harboring recombinant expression constructs containing promoters derived from the genome of mammalian cells (e.g., metallothionein promoter) or from mammalian viruses (e.g., the adenovirus late promoter; the vaccinia virus 7.5K promoter). In some embodiments, bacterial cells such as E. coli, and eukaryotic cells are used for the expression of entire recombinant antibody molecules. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express antibodies. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized to express a subject antibody. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). The efficiency of expression may be enhanced by the inclusion of appropriate transcription enhancer elements, transcription terminators, etc. (see Bittner et al., Methods in Enzymol. 153:51-544 (1987)).

For long-term, high-yield production of recombinant antibodies, stable expression may be used. For example, cell lines, which stably express the antibody molecule may be engineered. Rather than using expression vectors which contain viral origins of replication, host cells can be transformed with immunoglobulin expression cassettes and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into a chromosome and grow to form foci which in turn can be cloned and expanded into cell lines. Such engineered cell lines may be particularly useful in screening and evaluation of compounds that interact directly or indirectly with the antibody molecule.

A number of selection systems may be used, including but not limited to the herpes simplex virus thymidine kinase (Wigler et al., Cell 11:223 (1977)), hypoxanthine-guanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 Clinical Pharmacy 12:488-505; Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); TIB TECH 11(5):155-215 (1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981).

The host cell may be co-transfected with two expression vectors identified above, the first vector encoding the IgH chain and the second vector encoding the IgL chain. The two vectors may contain different selectable markers and origins of replication, which enable equal expression of heavy and light chain polypeptides. Alternatively, a single vector may be used which encodes, and is capable of expressing, both heavy and light chain polypeptides.

Once a subject engineered-antibody has been produced, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In some embodiments, antibodies are secreted from the cell into culture medium and harvested from the culture medium.

In some embodiments, 1, 2, 5, 10, 20, 50, 100, or more unique engineered antibodies may be produced by the subject method.

### Utility

A method for producing a library of engineered-antibody producing cells, compositions containing the same and compositions containing a plurality of engineered-antibodies as well as compositions containing individual engineered-antibodies is provided. Methods for screening the subject library, a plurality of engineered-antibodies, methods of identifying an engineered-antibody of interest, and methods for expressing an engineered-antibody of interest are provided. These methods and compositions have several uses, many of which will be described below.

In certain embodiments, an engineered-antibody produced by the methods provided herein may have a higher affinity, avidity, and/or specificity for the antigen compared to the two natural antibodies from which the VH and VL chains of the engineered-antibody were obtained.

In one embodiment, the invention provides methods of treating a subject with an engineered antibody of interest. In general these methods involve administering an engineered antibody identified by the methods described above to a host in need of treatment. In some embodiments, the engineered antibody is a therapeutic engineered antibody.

A variety of hosts are treatable according to the subject methods. Generally such hosts are mammals or mammalian, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, the hosts will be humans. In other embodiment, the host will be an animal model for a human disease.

Of particular interest is treatment and prevention of diseases, conditions and disorders associated with abnormal expression of a cellular protein, usually present on the surface of a cell, e.g. a cancer cell.

The methods and compositions provided herein have several research applications. In one exemplary application, the library of engineered antibodies is deposited onto an array or microarray (e.g. using a method provided by U.S. Pat. Nos. 6,372,483, 6,352,842, 6,346,416 and 6,242,266), and labeled samples (e.g. cell extracts or proteins) or pairs of differentially labeled samples are incubated with the array. Such experiments may provide antibodies and antibody-encoding polynucleotide sequences that differentially bind to samples. In one exemplary experiment, cancerous cells or extracts thereof are labeled and incubated with an array of engineered antibodies. After washing of the array, data representing the amount of binding of the cell or extract thereof may be extracted for each antibody. Comparison of this data to data generated using normal or non-cancerous cells incubated with a similar or the same array may reveal engineered antibodies that specifically recognize the cancer cell. Such antibodies have therapeutic applications.

The subject methods and compositions provide specific reagents that can be used in standard diagnostic procedures. For example, the subject antibodies or their immunoreactive fragments can be employed in immunoassays for detection of target antigens. To perform a diagnostic method, one of the subject compositions is provided as a reagent to detect a target antigen in a sample with which it reacts. Procedures for performing immunoassays are well established in the art and hence are not described here.

The engineered antibodies generated by the subject methods may also be used for treatment or prevention of diseases and conditions. The subject antibodies may be used to modulate the activities of target antigens that play a central role in disease development and/or progression. For example, a humanized anti-Her2 antibody, available commercially under the trademark HERCEPTIN®, which selectively inhibits growth of human breast cancer cells, is now employed as a potent drug to treat tens and thousands of breast cancer patients who overexpress the breast cancer antigen Her2.

### EXAMPLES

The following examples are provided in order to demonstrate and further illustrate certain embodiments and aspects of the present invention and are not to be construed as limiting the scope thereof.

### Example 1

### Method of Producing a Library of Engineered-Antibody Producing Cells Using Random Pairing

### Isolation of Antibody Producing Cells

Rabbits are immunized with an antigen using a standard immunization protocol. At about 10 days after the second booster immunization, antibody titers are determined using ELISA. Two booster immunizations are usually sufficient for obtaining high antibody titers. As soon as a high titer (detectable signal at 1:100000 dilution) is observed, the rabbit is sacrificed and bone marrow cells are collected from the femur and/or other large bones. Spleen cells and peripheral blood mononuclear cells (PBMCs) are also collected and frozen in 10% DMSO/90% FBS for analysis at a later time. Very large numbers of bone marrow cells (>2 billion) are obtained from a single rabbit. After washing, clearing of debris, and red-cell lysis, the antibody producing cells, which bind to the antigen with which the rabbit was immunized, are purified using FACS. Briefly, the antigen is conjugated to a fluorescent dye and the labeled antigen is incubated with the cells obtained above. The cells are briefly rinsed to wash off any antigen non-specifically attached to the cell. After rinsing, fluorescent cells are separated from unlabeled cells using FACS. These fluorescent cells express antibodies on their surface that specifically binds to the antigen with which the animal was immunized.

RT-PCR to Obtain IgH and IgL Chain cDNA

Primer Design:

In rabbit, the 5′ coding sequences of rabbit immunoglobulin heavy chain are primarily derived from only one gene. Antibody diversity is created by gene conversion and somatic mutation, but this does not affect the 5′ end of the antibody cDNA. Thus, most rabbit IgG H chains have very similar or identical signal peptide sequences, and the same is true for L chains. On the 3′ side, primers hybridizing to the constant domains, which also have identical sequences in most rabbit antibodies (rabbit constant domains are not divided into subclasses). As a result, only one pair of primers each is required for amplifying the vast majority of rabbit IgG H and L sequences. Typical priming sites are shown below, although any primer sites are used so long as the a variable domain-encoding polynucleotide is amplified. Typical primers for use with rabbit antibody-producing cells are as follows: heavy chain, 5′ end (CACCATGGAGACTGGGCTGCGCTGGCTTCTCCTGGTCGCTGTG (SEQ ID NO: 149)); heavy chain, 3′ end (CTCCCGCTCTCCGGGTAAATGAGCGCTGTGCCGGCGA (SEQ ID NO: 150)); light chain kappa, 5′ end (CAGGCAGGACCCAGCATGGACACGAGGGCCCCCACT (SEQ ID NO: 151)); and L kappa, 3′ end (TCAATAGGGGTGACTGTTAGAGCGAGACGCCTGC(SEQ ID NO: 152)).

Note that the 3′ H chain primer spans the 3′ end of the coding region, the stop codon, and the beginning of the 3′ UTR. Thus, this primer is specific for the secreted form of IgG, and does not recognize the transmembrane form, which does not contain this sequence due to alternative splicing. Therefore, the method is unlikely to recover IgG from memory B cells, which express predominantly the transmembrane form.

RT-PCR Conditions:

Cell lysis is done heating in a buffer containing RNAse inhibitors, followed by DNA degradation and reverse transcription performed at high temperature (60° C.) using a thermostable reverse transcriptase. Reverse transcription is primed by primers specific for the 3′ region of the IgG mRNAs. A single-step RT-PCR protocol is used, utilizing a thermostable enzyme that has both reverse transcriptase and DNA polymerase activities (MasterAmp™ RT-PCR Kit for High Sensitivity, Epicentre Technologies, Madison, Wis.). PCR products are analyzed by agarose gel electrophoresis. If required, a second round of PCR is performed with nested primers. In some PCR applications, this step is required to produce sufficient amounts of specific product.

Co-Amplification of H and L Chain cDNAs:

Different combinations of primers are tried, to accomplish efficient PCR amplification of H and L chain cDNAs in the same reaction. A ‘head start’ approach is often used, where PCR cycling is started with H chain primers alone; after a number of cycles (5 to 10) the L chain primers are added to the mix. Using these methods, similar yields of H and L chain are produced. Alternatively, a nested PCR approach is used for the H chain, by performing an initial round of PCR with primers amplifying the full-length cDNA, and a second round with primers amplifying only the vH-cH1-hinge portion of the H chain. This method should yield a product similar in size to the L chain cDNA. Expression of this product yields the F(ab′)2 fragment of IgG, which is divalent and fully active for antigen-binding.

IgG heavy and light chain PCR products are joined with CMV promoter and BGH3′pA (bovine growth hormone polyadenylation/transcription termination) sequences.

Method a) Overlap Extension PCR.

CMV Promoter Segment:

To prepare the CMV promoter fragment, the expression vector pcDNA-3 (which contains the CMV promoter and BGH3′pA segments) is used as a template, and the following PCR setup:

Primer 1 (5′ AATTCACATTGATTATTGAG 3′; SEQ ID NO: 153) corresponding to the 5′ end of the CMV promoter;

Primer 2 (5′ CAGCGCAGCCCAGTCTCCATCCCGTAAGCAGTGGGTTCTC 3′; SEQ ID NO: 154) corresponding to the 3′ end of the CMV promoter, and containing a 5′ extension (underlined) complementary to the 5′ end of the rabbit Ig H signal peptide sequence is performed.

PCR amplification with these primers produces a linear DNA fragment consisting of the CMV promoter (610 nt) and a 20 nt extension on the 3′ end, which is complementary to the 5′ end of the IgG vH coding region. As mentioned above, most rabbit IgGs contain 5′ vH (signal peptide) regions with nearly identical sequences. Therefore, only one primer pair is needed to amplify the majority of rabbit IgG cDNAs.

BGH3′pA Segment.

A similar approach is used to prepare the BGH3′pA segment. Again, the pcDNA3 expression vector is used as a template, and the following primers are used:

Primer 3 (5′ CCGGGTAAATGAGCGCTGTGGTTTAAACCCGCTGATCAGC 3′; SEQ ID NO: 155), corresponding to 5′ end of the BGH3′pA domain extended by a 20 nt sequence complementary to the 3′ end of the IgG heavy chain coding region, and including 11 nt of the 3′ untranslated domain.

Primer 4 (5′ AAGCCATAGAGCCGACCGCA 3′; SEQ ID NO: 156) corresponding to the 3′ end of the BGH polyadenylation domain.

PCR amplification results in a 250 nt fragment containing the BGH3′pA sequence and a 20 nt extension that overlaps with the 3′ end of the IgG heavy chain sequence.

Overlap Extension PCR:

The IgG heavy chain PCR product are mixed with the CMV promoter and BGH3′pA segments. The mixture is subjected to 10 cycles of PCR. The overlapping segments anneal, followed by extension of the overlapping 3′ ends. At the end of the 10 cycles, the outside primers (primers 1 and 4) are added to the mixture, and another 30 cycles of PCR are performed. The product is a 2100 nt fragment consisting of the CMV promoter, the IgG H coding sequence, and the BGH terminator.

IgG Light Chain:

The process are carried out in an analogous manner to produce 1500 nt fragments consisting of CMV promoter, kappa light chain coding sequence, and BGH terminator. A separate set of primers for lambda light chains can also be used to amplify and clone lambda light chains.

A low concentration of primers in the initial PCR reaction may be used. In some embodiments, primers are be designed such that amplification of the heavy chain results in a nucleotide encoding a form of the IgG H chain that is truncated at the 3′ end of the hinge domain. This fragment would be similar in size to the v kappa light chain. Co-expression of these fragments results in the secretion of F (ab′)₂ fragments of IgG.

Method b) Topoisomerase I Coupling.

This method is used as an alternative to overlap extension PCR. The overall experimental strategy is as described above. Commercially available topoisomerase-modified CMV promoter and BGH3′pA segments will be used (Invitrogen, San Diego, Calif.). The CMV promoter element (610 nt) is provided in a modified form with the topoisomerase recognition site (CCCTT) at its 3′ end, and a six base pair single-stranded overhang at the 3′ end (GCCTTG) which is used for directional coupling with the PCR product. The topoisomerase I enzyme is bound to the recognition site CCCTT. In order to be joined to the Topo-modified CMV promoter, the PCR product needs to contain the sequence CGGAACAAGGG (SEQ ID NO: 157) at its 5′ end. This sequence is cleaved by topoisomerase, resulting in a 6-base single-strand overhang that is complementary to the single-strand overhang of the CMV promoter element. These overhangs anneal and the fragments are covalently joined by the enzyme.

In order to link the IgG cDNA fragment to the CMV promoter, the 5′ primer used in the last round of IgG amplification are extended at its 5′ end with the sequence CGGAACAAGGG (SEQ ID NO: 158).

The linkage of the 3′ end of the IgG fragment with the BGH3′pA element is performed in an analogous manner, except that a different single-stranded overhang (GACTCA) is being used. This provides for directionality and selective joining of the 5′ end with the CMV promoter and the 3′ with the BGH terminator.

The joining reaction is carried out by mixing the 5′ CMV element, IgG PCR product, and 3′BGH element at a 1:2:1 ratio, and adding the 10× reaction buffer. The reaction proceeds rapidly and is usually complete within 10 min at room temperature. Following the reaction, a secondary PCR reaction is carried out, using primers corresponding to the 5′ end of the CMV promoter and the 3′ end of the BGH terminator (primers 1 and 4, see above). This results in the formation of the 2.1 kb IgG H expression cassette, or the 1.5 kb IgG L expression cassette. Conditions for co-production of H and L IgG expression cassettes in the same reaction are also envisioned.

The IgG H expression cassettes are cloned into a vector carrying a hygromycin resistance marker to generate an IgG H expression cassette library. The IgG L expression cassettes are cloned into a vector carrying a G418 resistance marker to generate an IgG L expression cassette library.

Equimolar amounts of the IgG H and IgG L expression cassette libraries are mixed and transfected into CHO cells. The transfected CHO cells are plated into 96-well or 384-well microtiter plates such that each well contains approximately one cell. Cells are maintained in media containing both hygromycin and G418. Cells that survive the double selection contain at least one expression cassette pair.

These cells are cultured and the antibodies produced by these cells are tested for binding to the antigen with which the rabbit was immunized.

### Example 2

### Related Antibodies

Antibodies were obtained from rabbit hybridoma cells producing anti-KDR antibodies that block the interaction of VEGF with its receptor (KDR). The hybridoma cells were generated by fusing immunized rabbit splenocytes with the rabbit hybridoma fusion partner 240E-W2.

New Zealand white rabbits were immunized with a fusion protein containing the rabbit Fc region and the extracellular domain of KDR. Each rabbit received a primary immunization by subcutaneous injection of 0.4 mg of the purified protein with complete Freund's or TiterMax adjuvant. The animals were then boosted by subcutaneous injection of 0.2 mg of the protein with incomplete Freund's or TiterMax once every three weeks. The final boost (0.4 mg protein in saline) was given intravenously 4 days before splenectomy.

Cell fusions were performed following the conventional protocol of Spieker-Polet using PEG. The ratio of splenocytes to the fusion partner was 2:1. The fused cells were plated in 96-well plates and HAT was added after 48 hrs to select for hybridomas. Direct ELISA was performed to identify antibodies that block binding of VEGF to a KDR fusion protein coated onto a microtiter plate. In this assay, the Fc-KDR ECD fusion protein was coated onto a 96-well ELISA plate and goat anti-rabbit IgG FEB conjugated to alkaline phosphatase was used to detect antibody binding to KDR. Antibodies identified in this assay were then were screened for blocking VEGF interaction with KDR in a ligand-receptor assay. The blocking antibodies were identified by their inhibition of binding of VEGF in solution to KDR coated on plates.

cDNAs coding the heavy and light chains of the antibodies were cloned and sequenced. The polypeptides encoded by the cDNAs were aligned and this alignment is shown in FIG. 2. FIG. 2 shows that two groups of related anti-KDR rabbit monoclonal Abs were obtained. Antibodies 69, 6, 71, 43, 81, 4, 30, 54, 57, 50, 68, 56, 83, 36, 77, 95, 14, 42, 27 belong to one group. Antibodies 2, 17, 3, 6, 9 belong to a different group.

FIG. 3 is a multiple sequence alignment of the H3 region of ten rabbit antibody sequences extracted from the Kabat database to illustrate the expected variation in unrelated antibodies.

### Example 3

### CDR-Anchored Amplification of Polynucleotides Encoding Related Antibodies

Several examples illustrating a method by which the amino acid sequences of related rabbit antibodies may be obtained by PCR are set forth in FIGS. 4A-4H. In the examples shown in FIGS. 4A-4D, reverse primers that are complementary to the CDR3 regions of the light chain of antibodies 31 (FIG. 4A), 29 (FIG. 4b), 27 (FIG. 4c) and 20 (FIG. 4d) were designed and can be used along with a universal forward primer (SEQ ID NO: 118) that binds to a site that is present in all rabbit antibody heavy chain sequences to amplify coding sequences for related antibodies. In the example shown in FIG. 4A, the primers designed against sequences that encode antibody 31 are expected to amplify light chain variable domain sequences for antibodies 11, 12, 2, 25, 22, 27, 3, 1, 19, 24, 23, 18, 13, 10 and 21, which are all from the same animal as antibody 31 and are related to antibody 31 by lineage. In the example shown in FIG. 4B, the primers designed against sequences that encode antibody 29 are expected to amplify light chain variable domain sequences for antibodies 8, 9, 16 and 32, which are all from the same animal as antibody 29 and are related to antibody 29 by lineage. In the example shown in FIG. 4C, the primers designed against sequences that encode antibody 27 are expected to amplify light chain variable domain sequences for other antibodies which are all from the same animal as antibody 27 and are related to antibody 27 by lineage. In the example shown in FIG. 4D, the primers designed against sequences that encode antibody 20 are expected to amplify light chain variable domain sequences for other antibodies which are all from the same animal as antibody 20 and are related to antibody 20 by lineage.

In the examples shown in FIGS. 4E-4H, reverse primers that are complementary to the CDR3 regions of the heavy chain of antibodies 31 (FIG. 4E), 29 (FIG. 4F), 29 (FIG. 4G) and 21 (FIG. 4H) were designed and can be used along with a universal forward primer (SEQ ID NO: 135) that binds to a site that is present in all rabbit antibody heavy chain sequences to amplify coding sequences for related antibodies. In the example shown in FIG. 4E, the primers designed against sequences that encode antibody 31 are expected to amplify heavy chain variable domain sequences for antibodies 2, 17, 22, 25, 12, 1, 24, 19, 25, 11, 31, 3, 10, 13, 21, 18 and 23, which are all from the same animal as antibody 31 and are related to antibody 31 by lineage. In the example shown in FIG. 4F, the primers designed against sequences that encode antibody 29 are expected to amplify heavy chain variable domain sequences for antibodies 8, 9, 16 and 32, which are all from the same animal as antibody 29 and are related to antibody 29 by lineage. In the example shown in FIG. 4G, the primers designed against sequences that encode antibody 27 are expected to amplify heavy chain variable domain sequences for other antibodies which are all from the same animal as antibody 27 and are related to antibody 27 by lineage. In the example shown in FIG. 4H, the primers designed against sequences that encode antibody 20 are expected to amplify heavy chain variable domain sequences for other antibodies which are all from the same animal as antibody 20 and are related to antibody 20 by lineage.

1-20. (canceled)

21. A library of cells that produce antibodies that comprise non-naturally paired IgH and IgL variable domains, wherein:
i. the sequences of the IgH and IgL variable domains are comprised by antibodies that are produced by an animal immunized with an antigen,
ii. the sequences of the IgH and IgL variable domains of are not paired together in said animal, and
iii. the antibodies in said cells bind to the same epitope in said antigen.

22. The library of claim 21, wherein library comprises at least 5 cells, each cell producing a different antibody.

23. The library of claim 22, wherein the cells that produce the different antibodies are in separate containers.

24. The library of claim 22, wherein the cells that produce the different antibodies are mixed together in the same container.

25. The library of claim 21, further comprising cells that produce naturally paired IgH and IgL variable domains.

26. The library of claim 21, wherein said animal is a rabbit.

27. The library of claim 21, wherein said cell is mammalian host cell.

28. The library of claim 21, wherein the IgH and IgL variable domains in said cells are from antibodies that are related by lineage in said animal.

29. The library of claim 21, wherein the IgH and IgL variable domains in said cells have CDR3s that have 0, 1 or 2 amino acid substitutions relative to one another.

30. The library of claim 21, wherein the antibodies produce by said cells comprise a heavy chain comprising said IgH variable domain and a light chain comprising said IgL variable domain.

31. The library of claim 21, wherein the antibodies produce by said cells are single chain antibodies.

32. The library of claim 21, wherein the IgH and IgL variable domains are encoded by a single vector in said cells.

33. The library of claim 21, wherein the IgH and IgL variable domains are encoded by a two vectors in said cells.
